(19) United States
(12) Patent Application Publication
(10) Pub. No.: US 2023/0392164 A1
(43) Pub. Date: Dec. 7, 2023

(54) DUAL EXPRESSION VECTOR FOR GENE AUGMENTATION FOR CRUMBS COMPLEX HOMOLOGUE 1 (CRB1) MUTATIONS

(71) Applicant: The Trustees of Columbia University in the City of New York, New York, NY (US)

(72) Inventors: Peter M.J. Quinn, New York, NY (US); Stephen H. Tsang, New York, NY (US); Yi-Ting Tsai, New York, NY (US)

(21) Appl. No.: 18/178,341

(22) Filed: Mar. 3, 2023

Related U.S. Application Data
(63) Continuation of application No. 17/555,284, filed on Dec. 17, 2021, now abandoned.
Application No. 17/555,284 is a continuation of application No. PCT/US2021/062925, filed on Dec. 10, 2021.
(60) Provisional application No. 63/123,766, filed on Dec. 10, 2020.

(51) Int. Cl. C12N 15/86; A61P 27/02; C07K 14/47

(52) U.S. Cl. CPC C12N 15/86; A61P 27/02; C07K 14/4702; A61K 48/00

(57) ABSTRACT

The present disclosure provides vectors comprising a transgene(s) encoding more than one isoform of Crumbs homologue-1 (CRB1) (e.g., CRB1-A and CRB1-B), and compositions thereof, for use in the treatment or prevention of CRB1-related diseases and disorder (e.g., autosomal recessive retinitis pigmentosa (RP) and Leber congenital amaurosis (LCA)).

### CROSS-REFERENCE TO RELATED APPLICATIONS

This application is a continuation of U.S. application Ser. No. 17/555,284, filed Dec. 17, 2021, which is a continuation of International Patent Application No. PCT/US2021/062925, filed Dec. 10, 2021, which claims the benefit of U.S. Provisional Application No. 63/123,766 filed Dec. 10, 2020, the contents of each of which are herein incorporated by reference in its entirety.

### SEQUENCE LISTING STATEMENT

The text of the computer readable sequence listing filed herewith, titled “COLUM-40032-303.XML”, created Aug. 22, 2023, having a file size of 11,680 bytes, is hereby incorporated by reference in its entirety.

### FIELD

The present disclosure provides vectors and compositions for treating or preventing CRB1-related diseases and disorder.

### BACKGROUND

The Crumbs complex (CRB) is crucial for cell polarity and epithelial tissue function, having an essential role during retinogenesis. Disruption of the CRB complex will interrupt the precise orchestration of spatiotemporal process during retinal development, such as cell fate choice, division, migration, and differentiation. This can cause retinal degeneration leading to impairment of retinal function and thus vision.

Mutations in the Crumbs homologue-1 (CRB1) gene cause progressive and disabling autosomal recessive retinal dystrophies. Approximately 80,000 patients are affected worldwide, with a prevalence in the United States of 1 in 86,500. CRB1 mutations exhibit high phenotypic variability, with approximately 310 pathogenic variants reported. According to a meta-analysis, CRB1 gene mutations account for 2.7% and 10.1% of autosomal recessive retinitis pigmentosa (RP) and Leber congenital amaurosis (LCA) cases, respectively, as well as an increasing number of juvenile macular dystrophy cases. LCA is a group of severe, infantile-onset retinal dystrophies that constitute more than 5% of all retinal dystrophies and is the most common cause of inherited blindness in childhood. Despite its prevalence and severity, the pathogenesis of CRB1 LCA remains unclear, and there is no treatment available to date.

Despite the large number of pathogenic CRB1 mutations identified, there is still no clear genotype-phenotype correlation. This may be due to the existence of multiple isoforms of CRB1 with cell type-specific roles.

There is an urgent need in the art for compositions and methods that can be used to treat a subject that has or is at risk of developing a disease characterized by Crumbs homologue 1 (CRB1) mutations.

### SUMMARY

The present disclosure relates to compositions and methods that can be used to treat a subject (e.g., a mammalian subject, such as a human subject) that has or is at risk of developing a disease characterized by Crumbs homologue 1 (CRB1) mutations including but not limited to autosomal recessive retinitis pigmentosa (RP) and Leber congenital amaurosis (LCA).

Using the compositions and methods of the disclosure, a subject (e.g., a mammalian subject, such as a human subject) that has or is at risk of developing a disease described above may be administered a composition containing a transgene encoding one or more of the foregoing proteins. The composition may be a vector, for example, a viral vector, such as a lentivirus vector.

In one embodiment, the disclosure features a method of treating, preventing, and/or curing a disease or disorder characterized by CRB1 mutations including but not limited to autosomal recessive retinitis pigmentosa (RP) and Leber congenital amaurosis (LCA) in a subject in need thereof. In some embodiments, the method includes administering to the subject a therapeutically effective amount of a composition containing, comprising, or consisting essentially of transgenes encoding more than one isoform of CRB1 (e.g., CRB1-A, CRB1-B, CRB1-C). In some embodiments, the composition contains, comprises, or consists essentially of a transgene encoding CRB1-A and a transgene encoding CRB1-B. In some embodiments, the method includes administering to a subject in need thereof a therapeutically effective amount of a composition, such as a viral vector, comprising a nucleic acid encoding an isoform of CRB1, such as CRB1-A or CRB1-B, configured to allow expression of the CRB1 isoform in more than one retinal cell type (e.g., Müller glial cells and photoreceptor cells).

In an additional embodiment, the disclosure features a method of alleviating one or more symptoms associated with a disease or disorder characterized by CRB1 mutations including but not limited to autosomal recessive retinitis pigmentosa (RP) and Leber congenital amaurosis (LCA) in a subject in need thereof. The method includes administering to the subject a therapeutically effective amount of a composition containing, comprising, or consisting essentially of transgenes encoding more than one isoform of CRB1. In some embodiments, the composition contains, comprises, or consists essentially of a transgene encoding CRB1-A and a transgene encoding CRB1-B.

The disclosure also provides a composition containing, comprising, or consisting essentially of transgenes encoding more than one isoform of CRB1. In some embodiments, the composition contains, comprises, or consists essentially of a transgene encoding CRB1-A and a transgene encoding CRB1-B.

In some embodiments of the disclosure, the composition comprises a vector encoding more than one isoform of CRB1, such as a viral vector, or a combination of vectors which collectively encode more than one isoform of CRB1. The viral vector(s) may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule).

In some embodiments, the more than one isoform of CRB1 comprises CRB1-A and CRB1-B.

In some embodiments, the transgenes encoding more than one isoform of CRB1 are provided on a single vector. In some embodiments, the single vector is a viral vector. In some embodiments, the viral vector is derived from a virus selected from the group consisting of adeno-associated virus (AAV), adenovirus, lentivirus, retrovirus, poxvirus, baculovirus, herpes simplex virus, vaccinia virus, and a synthetic virus. In some embodiments, the viral vector is derived from lentivirus. In some embodiments, the transgenes encoding more than one isoform of CRB1 are operably linked to the same or different promoter.

In some embodiments, the transgenes encoding more than one isoform of CRB1 are provided on two or more vectors. In some embodiments, the two or more vectors are each individually derived from a virus selected from the group consisting of adeno-associated virus (AAV), adenovirus, lentivirus, retrovirus, poxvirus, baculovirus, herpes simplex virus, vaccinia virus, and a synthetic virus. In some embodiments, at least one or both of the two or more vectors are derived from adeno-associated virus. In some embodiments, the transgenes encoding more than one isoform of CRB1 are operably linked to the same or different type promoter.

In some embodiments, at least one or all of the more than one isoform of CRB1 is operably linked to a tissue-specific or cell type-specific control or regulatory element. In some embodiments, any or all of the more than one isoform of CRB1 are operably linked to constitutive promoter.

In some embodiments, CRB1-A is operably linked to a promoter which induces expression in Milner glial cells. In some embodiments, the promoter which induces expression in Müller glial cells is selected from the group consisting of RLBP1, GfaABC1D, GFAP, ProB2 and PROC17.

In some embodiments, the CRB1-B is operably linked to a promoter which induces expression in photoreceptor cells. In some embodiments, the promoter which induces expression in photoreceptor cells is selected from the group consisting of IRBP, CAR, RHO, PR1.7, ProA1, ProA6, ProC1, ProA14, ProA36 and GRK1.

In some embodiments of the disclosure, the transgenes are operably linked to separate promoters that induce expression of the transgenes in the proper cells, e.g., CRB1-A predominately in a Müller glial cells and CRB1-B in photoreceptor cells.

In certain embodiments, the route of administration is subretinal injection or intravitreal injection.

The disclosure also provides a recombinant viral vector comprising a transgene encoding more than one isoform of Crumbs homologue-1 (CRB1). In some embodiments, the more than one isoform of CRB1 comprises CRB1-A and CRB1-B. In some embodiments, viral vector is derived from a virus selected from the group consisting of adeno-associated virus (AAV), adenovirus, lentivirus, retrovirus, poxvirus, baculovirus, herpes simplex virus, vaccinia virus, and a synthetic virus. In some embodiments, the viral vector is derived from a lentivirus.

In some embodiments, CRB1-A is operably linked to a promoter which induces expression in Müller glial cells. In some embodiments, the promoter which induces expression in Müller glial cells is selected from the group consisting of RLBP1, GfaABC1D, GFAP, ProB2 and PROC17.

In some embodiments, the CRB1-B is operably linked to a promoter which induces expression in photoreceptor cells. In some embodiments, the promoter which induces expression in photoreceptor cells is selected from the group consisting of IRBP, CAR, RHO, PR1.7, ProA1, ProA6, ProC1, ProA14, ProA36 and GRK1.

In some embodiments, the more than one isoforms of CRB1 are operably linked to a promoter selected from the group consisting of CMV, EF1, CAG, CB7, PGK and SFFV.

In some embodiments, the vector further comprises a polyadenylation sequence (e.g., SV40, bGHpolyA and spA), a post-transcriptional regulatory element (e.g., WPRE, WPRE3 and HPRE), or combinations thereof.

Also provided are compositions comprising the recombinant vector.

In some embodiments, the compositions disclosed herein may further comprise a pharmaceutical carrier or vehicle. In some embodiments, the compositions are suitable for subretinal injection or intravitreal injection.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For the purpose of illustrating the invention, there are depicted in drawings certain embodiments of the invention. However, the invention is not limited to the precise arrangements of the embodiments depicted in the drawings.

FIG. 1 shows the isoform distribution based on 317 unique CRB1 patient mutations.

FIGS. 2A-2H show the CRB1 isoform diversity in the human retina. Images of BaseScope staining of human adult retina (FIGS. 2A, 2C, 2E, 2G) and hiPSC-derived retinal organoids (FIGS. 2B, 2D, 2F, 2H) using isoform-specific probes (red), and nuclear counterstain using Gill's Hematoxylin 1 (blue) are shown. PAN-CRB1 probe shown in FIGS. 2A and 2B. The CRB1-A probe is shown in FIGS. 2C and 2D. The CRB1-B probe is shown in FIGS. 2E and 2F. The CRB1-C probe is shown in FIGS. 2G and 2H.

FIG. 3 is a schematic of the CRB1 Isoform Dual Expression Vector and a schematic of a retina showing targeting of CRB1-A to Müller glial cells and CRB1-B to photoreceptor cells.

FIGS. 4A-4F show lentiviral and AAV transduction of retinal organoids (ROs). FIGS. 4A and 4B show day 145 WT RO, 5 days post-transfection with Lenti-EF1-GFP (green). Transduction of ONL and INL. RO infected with AAV8-CMV-GFP (green) at DD140 (C—F) analyzed 16 days later (DD156). FIG. 4C is a brightfield image showing GFP expression in late RO, segments can be detected (arrowheads). FIG. 4D shows GFP expression is found in both the ONL and INL). FIG. 4E shows GFP expression overlaps with recoverin staining (red) in the ONL (arrowheads). FIG. 4F shows GFP expression overlaps with Sox9 staining (red) in the INL (arrowheads). GCL=ganglion cell layer; INL=inner nuclear layer; ONL=outer nuclear layer.

FIGS. 5A-5G show CRB1 lentiviral vectors for gene augmentation in ROs. Immunoblots against GFP (FIG. 5A), FLAG (FIG. 5B), and CRB1 (FIG. 5C) from HEK293 cells transduced with Lenti-CRB1-P2A-GFP (FIGS. 5A,C) and Lenti-CRB1-FLAG (FIGS. 5B,C) compared to untransduced cells (CTRL). In FIG. 5A, the unfused GFP fragment is found. Full-length CRB1 is 153 kDa. In FIG. 5B, the FLAG-tagged fusion to CRB1 is found at the correct molecular weight. FIGS. 5C, D show CRB1 overexpression with the Lenti-CRB1-GFP and -FLAG vectors. In FIGS. 5E,F Lenti-CRB1-GFP is expressed in Müller glial cells (MGCs) (Sox9+) with identifiable apical and basal processes. In FIG. 5G, mature photoreceptor cells (PRCs) (Recoverin+) are also transduced.

FIG. 6 shows the phenotype of failure in biosynthesis of photoreceptor outer segments in CRB1 patient retinal organoids versus wild-type control.

FIGS. 7A-7C show the strategy for the generation of CRB1 knockout iPSC. FIGS. 7A and 7B are schematics of targeting strategy. sgRNA1—SEQ ID NO: 1; sgRNA2—SEQ ID NO: 2; sgRNA3—SEQ ID NO: 3; sgRNA4—SEQ ID NO: 4; CRB1 Exon 5—SEQ ID NO: 5; and CRB1 Exon 7—SEQ ID NO: 6. FIG. 7C shows in vitro cleavage of target DNA by RNP.

FIGS. 8A-8D show the generation of CRB1 knockout iPSC. FIG. 8A is a schematic of a dual guide CRB1 Null construct. FIG. 8B is a graph of ICE (Inference of CRISPR Edits) analysis of cutting efficiency in HEK293 and two iPSC lines. Shown below are guide (SEQ ID NO: 1) and PAM sequences. FIG. 8C shows confirmation of deletion in HEK293 and two iPSC lines. sgRNA1—SEQ ID NO: 1; sgRNA3—SEQ ID NO: 3; and portions of CRB1 Exon 5—SEQ ID NO: 5; and CRB1 Exon 7—SEQ ID NO: 6. FIG. 8D shows that the predominately amplified sequence (SEQ ID NO: 7) introduces premature stop codon for nonsense mediated decay.

FIG. 9 shows an increased outer nuclear layer (ONL) thickness in CRB1 LCA patient Retinal Organoids compared to controls by immunohistochemistry staining for recoverin (a photoreceptor marker) of control vs patient retinal organoids at differentiation day 90. Below shows quantification of the thickness of the outer nuclear layer (ONL, where the photoreceptors are), control retinal organoids against two clones (ASD/AS4) from a patient with a homozygous 1103 mutation who has LCA.

FIG. 10 shows images of CRB1 Isoforms in human cadaveric retina and retinal organoids. Antibodies specifically targeting each CRB1 retinal isoform were synthesized. CRB1-A antibody localizes to the subapical region (arrowhead). CRB1-B localizes to the PRC segments (arrowhead) and seems to localize most strongly to cones (asterisk). CRB1-C appears to localize to nuclei in the INL (asterisk), the synaptic layer, and PRC segments (arrowhead).

FIGS. 11A-11E show equine infectious anemia virus (EIAV)-based lentivirus single vector dual promoter reporter. FIG. 11A is a schematic of a EIAV single vector dual promoter (SVDP) construct. In-tandem, Human IRBP promoter drives mCherry expression and the human RLBP1 promoter drives eGFP expression. Transfection of EIAV-SVDP in HEK293 cells shows dual expression by immunofluorescence (FIG. 11B) and by immunoblot (FIGS. 11C and 11D) in comparison to untransduced HEK293 controls (CTRL). Viral particles produced from EIAV-SVDP show dual expression by immunofluorescence in HEK293 cells (FIG. 11E).

FIGS. 12C-12D are schematics of exemplary human immunodeficiency virus (HIV)-based lentivirus single vector dual promoter construct driving CRB1-A and CRB1-B expression. FIG. 12A is an exemplary in tandem design with hIRBP promoter driving a codon-modified CRB1-B and a minimal CMV promoter driving a codon-modified CRB1-A. FIG. 12B is an exemplary bi-directional design with hIRBP promoter driving a codon-modified CRB1-B and a minimal CMV promoter driving a codon-modified CRB1-A. FIG. 12C is an exemplary in tandem design with hIRBP promoter driving a codon-modified CRB1-B and a RLBPlpromoter driving a codon-modified CRB1-A. FIG. 12D is an exemplary bi-directional design with hIRBP promoter driving a codon-modified CRB1-B and a RLBPlpromoter driving a codon-modified CRB1-A.

FIGS. 13A and 13B show photoreceptor specific promoters drive expression of fluorescent reporters in human Retinal Organoids. FIG. 13A shows retinal organoids transduced with EIAV single vector dual promoter (SVDP) construct express mCherry in the outer nuclear layer. FIG. 13B shows retinal organoids transduced with AAV8.hGRK1.GFP express GFP in the outer nuclear layer and co-localization with recoverin a photoreceptor maker.

FIGS. 14A-14E show exemplary AAV constructs for CRB1-A (FIG. 14A—pAAV.sCMV.CRB1A. SPA; FIG. 14B—pAAV.sCMV.CRB1A.FLAG. SPA), CRB1-B (FIG. 14D—pAAV.sCMV.CRB1B.P2A.mKO2.CWSL3; FIG. 14E—pAAV.sCMV.CRB1B.HIS6.P2A.mK02.CWSL3), and GFP (FIG. 14C).

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recitation of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” means within an acceptable error range for the particular value as determined by one of ordinary skill in the art, which will depend in part on how the value is measured or determined, e.g., the limitations of the measurement system, e.g., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “subject” as used in this application refers to animals in need of therapeutic or prophylactic treatment. Subjects include mammals, such as canines, felines, rodents, bovine, equines, porcines, ovines, and primates. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is known or suspected of having a disease or disorder characterized by CRB1 mutations including but not limited to autosomal recessive retinitis pigmentosa (RP) and Leber congenital amaurosis (LCA).

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat,” “treatment,” and the like refer to a means to slow down, relieve, ameliorate, or alleviate at least one of the symptoms of the disease or disorder, or reverse the disease or disorder after its onset.

The terms “prevent,” “prevention,” and the like refer to acting prior to overt disease or disorder onset, to prevent the disease or disorder from developing or minimize the extent of the disease or disorder, or slow its course of development.

The term “cure” and the like means to heal, to make well, or to restore to good health or to allow a time without recurrence of disease so that the risk of recurrence is small.

The term “in need thereof” would be a subject known or suspected of having or being at risk of having a disease or disorder characterized by CRB1 mutations including but not limited to autosomal recessive retinitis pigmentosa (RP) and Leber congenital amaurosis (LCA).

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically-acceptable” refers to molecular entities and compositions that do not produce an allergic or similar untoward reaction when administered to a host, such as gastric upset, dizziness, and the like, when administered to a human, and approved by a regulatory agency of the Federal or a state government or listed in the U.S. Pharmacopeia or other generally recognized pharmacopeia for use in animals, and more particularly in humans.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of viral vector. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA from a transcribed gene.

Standard methods in molecular biology are described Sambrook, Fritsch and Maniatis Molecular Cloning, A Laboratory Manual, Cold Spring Harbor Laboratory Press, Cold Spring Harbor, NY (1982 & 1989 2nd Edition, 2001 3rd Edition); Sambrook and Russell Molecular Cloning, 3rd ed., Cold Spring Harbor Laboratory Press, Cold Spring Harbor, N Y (2001); Wu Recombinant DNA, Vol. 217, Academic Press, San Diego, CA) (1993). Standard methods also appear in Ausbel, et al. Current Protocols in Molecular Biology, Vols. 1-4, John Wiley and Sons, Inc. New York, NY (2001).

Crumbs (Crb) is a large transmembrane protein initially discovered at the apical membrane of Drosophila epithelial cells (Tepass et al., 1990). The human CRB1 gene is mapped to chromosome 1q31.3, and contains 12 exons, has 12 identified transcript variants so far, three CRB family members, and over 210 kb genomic DNA (Den Hollander et al., 1999). Canonical CRB1 (CRB1-A) is a large transmembrane protein consisting of multiple epidermal growth factor (EGF) and laminin-globular like domains in its extracellular N-terminus. The intracellular C-terminal domain contains a FERM and a conserved glutamic acid-arginine-leucine-isoleucine (ERLI) PDZ binding motives. An alternative transcript of CRB1, CRB1-B, was recently described and suggested to have significant extracellular domain overlap with canonical CRB1 while bearing unique 5′ and 3′ domains (Ray et al., 2020). In mammals, CRB1 is a member of the Crumbs family together with CRB2 and CRB3.

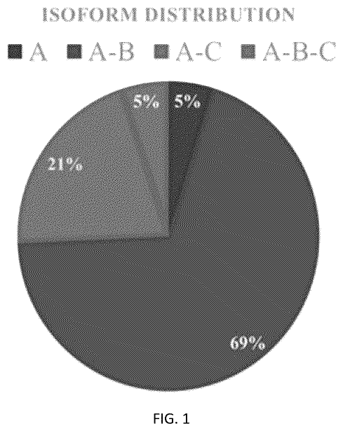

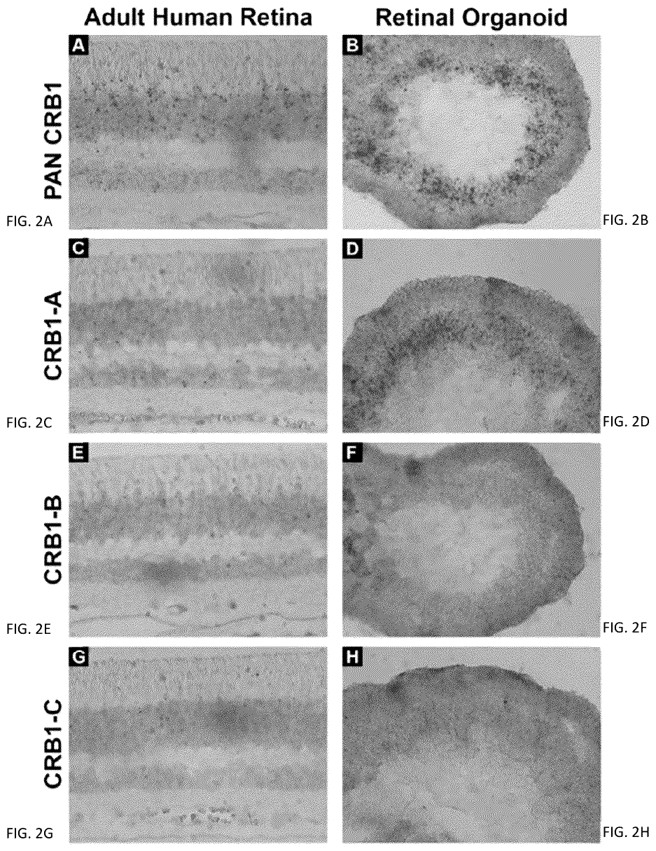

CRB is localized in the retina. The current disclosure is based upon the finding that CRB1-A is predominantly expressed in Müller glial cells and CRB1-B in photoreceptor cells as well as the fact that approximately 70% of novel CRB1 patient mutations affect both CRB1-A and CRB1-B. See FIGS. 1 and 2.

The current disclosure is based, at least in part, upon this discovery that CRB isoforms are distributed in different cell types in the human retina. These findings mean that diseases characterized by CRB1 mutations can be treated, prevented and/or cured via gene therapy by delivery of nucleic acids encoding more than one isoform of CRB1 in one viral vector (denoted CRB1 Isoform Dual Expression Vector) or composition comprising one or more nucleic acids encoding more than one isoform of CRB1.

### Transgene Coding Sequences

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In embodiments of the current invention the transgenes would encode a functional protein including but not limited to CRB1-A and CRB1-B.

The human CRB1-A gene (GenBank: MT470365.1) can be used to obtain a transgene.

In some embodiments, the transgene encodes CRB1-A. In some embodiments, the transgene encodes human CRB1-A (SEQ ID NO: 8). The CRB1-A may have an amino acid sequence that is at least 85% identical to the amino acid sequence of human CRB1-A (SEQ ID NO: 8) (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human CRB1-A). In some embodiments, the CRB1-A has an amino acid sequence that is at least 90% identical to the amino acid sequence of human CRB1-A (e.g., an amino acid sequence that is 90%, 91%, 92%, 93%, 94%, 95%, 96%, 97%, 98%, 99%, or 100% identical to the amino acid sequence of human CRB1-A). In some embodiments, the CRB1-A has an amino acid sequence that is at least 95% identical to the amino acid sequence of human CRB1-A (e.g., an amino acid sequence that is 95%, 96%, 97%, 98%, 99%, or 100% identical to the amino acid sequence of human CRB1-A).

In some embodiments, the CRB1-A has an amino acid sequence that differs from human CRB1-A (SEQ ID NO: 8) by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the CRB1-A has an amino acid sequence that differs from human CRB1-A (SEQ ID NO: 8) by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding CRB1-A has a nucleic acid sequence that is at least 70% identical to the nucleic acid sequence of human CRB1-A (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human CRB1-A). In some embodiments, the transgene encoding CRB1-A has a nucleic acid sequence that is at least 85% identical to the nucleic acid sequence of human CRB1-A (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human CRB1-A). In some embodiments, the transgene encoding CRB1-A has a nucleic acid sequence that is at least 90% identical to the nucleic acid sequence of human CRB1-A (e.g., a nucleic acid sequence that is 90%, 91%, 92%, 93%, 94%, 95%, 96%, 97%, 98%, 99%, or 100% identical to the nucleic acid sequence of human CRB1-A). In some embodiments, the transgene encoding CRB1-A has a nucleic acid sequence that is at least 95% identical to the nucleic acid sequence of human CRB1-A (e.g., a nucleic acid sequence that is 95%, 96%, 97%, 98%, 99%, or 100% identical to the nucleic acid sequence of human CRB1-A).

In some embodiments, the transgene encoding CRB1-A is codon optimized to increase efficiency.

The human CRB1-B gene (GenBank: MT47036) can be used to obtain a transgene.

In some embodiments, the transgene encodes CRB1-B. In some embodiments, the transgene encodes human CRB1-B (SEQ ID NO: 9). The CRB1-B may have an amino acid sequence that is at least 85% identical to the amino acid sequence of human CRB1-B (SEQ ID NO: 9) (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human CRB1-B). In some embodiments, the CRB1-B has an amino acid sequence that is at least 90% identical to the amino acid sequence of human CRB1-B (e.g., an amino acid sequence that is 90%, 91%, 92%, 93%, 94%, 95%, 96%, 97%, 98%, 99%, or 100% identical to the amino acid sequence of human CRB1-B). In some embodiments, the CRB1-B has an amino acid sequence that is at least 95% identical to the amino acid sequence of human CRB1-B (e.g., an amino acid sequence that is 95%, 96%, 97%, 98%, 99%, or 100% identical to the amino acid sequence of human CRB1-B).

In some embodiments, the CRB1-B has an amino acid sequence that differs from human CRB1-B (SEQ ID NO: 9) by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the CRB1-B has an amino acid sequence that differs from human CRB1-B by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding CRB1-B has a nucleic acid sequence that is at least 70% identical to the nucleic acid sequence of human CRB1-B (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human CRB1-B). In some embodiments, the transgene encoding CRB1-B has a nucleic acid sequence that is at least 85% identical to the nucleic acid sequence of human CRB1-B (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human CRB1-B). In some embodiments, the transgene encoding CRB1-B has a nucleic acid sequence that is at least 90% identical to the nucleic acid sequence of human CRB1-B (e.g., a nucleic acid sequence that is 90%, 91%, 92%, 93%, 94%, 95%, 96%, 97%, 98%, 99%, or 100% identical to the nucleic acid sequence of human CRB1-B). In some embodiments, the transgene encoding CRB1-B has a nucleic acid sequence that is at least 95% identical to the nucleic acid sequence of human CRB1-B (e.g., a nucleic acid sequence that is 95%, 96%, 97%, 98%, 99%, or 100% identical to the nucleic acid sequence of human CRB1-B).

In some embodiments, the transgene encoding CRB1-B is codon optimized to increase efficiency.

It will be appreciated that changing native codons to those most frequently used in mammals allows for maximum expression in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons.

Vectors

In some embodiments of the disclosure, the composition comprises a vector or vectors, such as viral vectors, encoding transgenes of one or more CRB1 isoforms. The viral vector(s) may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule).

In one embodiment, the vector or vectors are derived from a lentivirus. Lentiviral vectors are part of a larger group of retroviral vectors. A detailed list of lentiviruses may be found in Coffin et al. (1997) “Retroviruses” Cold Spring Harbor Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” Visna Maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV).

In some embodiments, the lentiviral vector is EIAV based.

In some embodiments, the lentiviral vector is HIV based. The HIV based vector may be an HIV-1, or HIV-2 based vector, such as a vector derived from HIV-1M, for example, from the BRU or LAI isolates.

Details on the genomic structure of some lentiviruses may be found in the art. By way of example, details on HIV and EIAV may be found from the NCBI Genbank database (e.g., Genome Accession Nos. AF033819 and AF033820 respectively). Details of HIV variants may also be found in the HIV databases maintained by Los Alamos National Laboratory. Details of EIAV clones may be found at the NCBI database maintained by the National Institutes of Health. Also see U.S. Pat. Nos. 7,790,419; 7,585,676; 7,419,829; 7,351,585; 7,303,910; 7,198,784; 7,070,994; 6,924,123; 6,818,209; 6,808,922; 6,800,281; 6,783,981; 6,541,248; 6,312,683; and 6,312,682.

A lentiviral vector, as used herein, is a vector which comprises at least one component part derivable from a lentivirus. That component part may be involved in the biological mechanisms by which the vector infects cells, expresses genes, or is replicated.

In some optional embodiments, vectors of the present invention are recombinant lentiviral vectors. The term “recombinant lentiviral vector” refers to a vector with sufficient lentiviral genetic information to allow packaging of an RNA genome, in the presence of packaging components, into a viral particle capable of infecting a target cell. Infection of the target cell may include reverse transcription and integration into the target cell genome. The recombinant lentiviral vector carries non-viral coding sequences which are to be delivered by the vector to the target cell. A recombinant lentiviral vector is incapable of independent replication to produce infectious lentiviral particles within the final target cell. Usually, the recombinant lentiviral vector lacks a functional gag-pol and/or env gene and/or other genes essential for replication. Optionally, the recombinant lentiviral vector of the present invention has a minimal viral genome. As used herein, the term “minimal viral genome” means that the viral vector has been manipulated so as to remove the non-essential elements and to retain the essential elements in order to provide the required functionality to infect, transduce and deliver a nucleotide sequence of interest to a target host cell.

In one embodiment, the vector or vectors are derived from or based on adeno-associated viruses (AAVs). Adeno-associated viruses (AAV), from the parvovirus family, are small viruses with a genome of single stranded DNA. Because AAV are not associated with pathogenic disease in humans, AAV vectors are able to deliver therapeutic proteins and agents to human patients without causing substantial AAV pathogenesis. The adeno-associated virus may be of any serotype, a mixture of serotypes, or variants thereof. Exemplary AAV serotypes include AAV1, AAV 2, AAV3, AAV4, AAV5, AAV6, AAV7, AAV8, AAV9, AAV10, and AAV11. For example, when the viral transfer vector is based on a mixture of serotypes, the viral transfer vector may contain the capsid signal sequences taken from one AAV serotype (for example selected from any one of AAV serotypes 1, 2, 3, 4, 5, 6, 7, 8, 9, 10, and 11) and packaging sequences from a different serotype (for example selected from any one of AAV serotypes 1, 2, 3, 4, 5, 6, 7, 8, 9, 10, and 11).

An AAV vector, as used herein, is a vector which comprises at least one component part derivable from adeno-associated viruses. That component part may be involved in the biological mechanisms by which the vector infects cells, expresses genes, or is replicated. In some embodiments, all or a part of the viral genome has been replaced with a transgene, which is a non-native nucleic acid with respect to the AAV nucleic acid sequence. AAV vectors generally have had up to approximately 96% of the parental genome deleted, such that only the terminal repeats (ITRs), which contain recognition signals for DNA replication and packaging, remain. Thus, the AAV vector may be a recombinant AAV vector.

For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757.

In some embodiments, the vector(s) may be configured or modified to confer increased infectivity of one or more types of cells. In the case of two or more vectors, each vector may be configured to confer increased infectivity in the same or different cell types. In some embodiments, the vector(s) may be configured to confer increased infectivity in one or more types of retinal cells (e.g., a photoreceptor cell (e.g., rods; cones), a retinal ganglion cell (RGC), a glial cell (e.g., a Müller glial cell, a microglial cell), a bipolar cell, an amacrine cell, a horizontal cell, and/or a retinal pigmented epithelium (RPE) cell). See for example, International Patent Publication No. WO2019104279, incorporated herein by reference in its entirety.

Due to size constraints of viral genomes for packaging, the transgenes of more than one isoform of CRB1 can be engineered and packaged in two or more vectors/stocks. Whether packaged in one vector or stock which is used as a composition according to the invention, or in two or more vectors or stocks which form a virus composition of the invention, the composition collectively contains the transgenes of more than one isoform of CRB1.

In some embodiments, the composition comprises two vectors, e.g., two viral vectors, each encoding a transgene of at least one CRB1 isoform. For example, the composition may comprise a first vector encoding a first CRB1 isoform and a second vector encoding a second CRB1 isoform. In some embodiments, the composition comprises a first vector encoding a CRB1-A transgene and a second vector encoding a CRB1-B transgene, as described herein.

In some embodiments, the two viral vectors are derived from the same or different virus. For example, the two viral vectors may each be AAV-based vectors or lentivirus-based vectors. Alternatively, the first vector may be an AAV-based vector and the second vector may be a lentivirus-based vectors.

In some embodiments, the viral vector may further comprise sequences which facilitate packaging into a viral vector, such as AAV inverted terminal repeats (ITRs) or lentiviral long terminal repeats (LTRs.)

In some embodiments, the viral vector further comprises an enhancer, such as the CMV enhancer.

In some embodiments, the viral vector further comprises an antibiotic resistance marker (e.g., AmpR).

In some embodiments, a dual expression vector further comprises a sequence encoding a reporter, such as a sequence encoding a fluorescent protein (e.g., GFP, mCherry, Kusabira-Orange).

The vectors may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter, and enhancer sequences; efficient RNA processing signals such as splicing and polyadenylation (polyA) or synthetic polyA (SPA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide.

For example, commonly used promoters are derived from polyoma, adenovirus 2, cytomegalovirus, simian virus 40, and others disclosed herein and known in the art. Vectors of the present disclosure can comprise any of a number of promoters known to the art, wherein the promoter is constitutive, regulatable or inducible, cell type specific, tissue-specific, or species specific. Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), CB7 (chicken β-actin promoter) and the like. Additional promoters, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

The vectors of the present disclosure may direct expression of the nucleic acid in a particular cell type. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

In some embodiments of the disclosure, the transgenes are operably linked to separate promoters that induce expression of the transgenes in the proper cells, e.g., CRB1-A in Müller glial cells and CRB1-B in photoreceptor cells.

The promoter for CRB1-A may be, for example, RLBP1 (Retinaldehyde Binding Protein 1), GFAP (Glial fibrillary acidic protein), GfaABC1D (a truncated GFAP promoter), and synthetic promoters ProB2 and PROC17.

The promoter for CRB1-B may be, for example, interphotoreceptor retinoid-binding protein (IRBP), cone arrestin (CAR), rhodopsin (RHO), PR1.7 (a truncated version of version of the L-opsin promoter), synthetic promoters: ProA1, ProA6, ProC1, ProA14, and ProA36, and G protein-coupled receptor kinase 1 (GRK1).

Alternatively, ubiquitous promoters may be used including without limitation, CMV, EF1, CAG, CB7, PGK, and SFFV.

Other regulatory elements may also be used such as a polyadenylation sequence and post-transcriptional regulatory elements, for efficient pre-mRNA processing and increasing gene expression, respectively. For nucleic acids encoding proteins, a polyadenylation sequence generally is inserted following the transgene sequences. Examples of polyadenylation sequences include SV40, bGHpolyA, and spA. Examples of post-transcriptional regulatory elements include WPRE, WPRE3, and HPRE.

In some embodiments, optimized combinations of polyadenylation sequences and post-transcriptional regulatory elements, such as CWSL3, may be used in the vectors (Choi et al. 2014).

The precise nature of the regulatory sequences needed for gene expression in host cells may vary between species, tissues, or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

In some embodiments, the regulatory sequences impart tissue-specific gene expression capabilities. In some cases, the tissue-specific regulatory sequences bind tissue-specific transcription factors that induce transcription in a tissue specific manner.

In some embodiments of the disclosure, the composition comprises one or more viral vectors, collectively encoding two or more CRB1 isoforms, wherein one or more of the two or more CRB1 isoforms are operably linked to a tissue-specific or cell type-specific control or regulatory element (e.g., a promoter). In some embodiments, each of the two or more CRB1 isoforms are operably linked to the same or different tissue-specific or cell type-specific control or regulatory element. In some embodiments, the two or more CRB1 isoforms comprise CRB1-A and CRB1-B.

In some embodiments, the composition comprises a single viral vector, encoding CRB1-A and CRB1-B, wherein one or both of CRB1-A and CRB1-B are operably linked to a tissue-specific or cell type-specific control or regulatory element. In some embodiments, the composition comprises a single viral vector, encoding CRB1-A and CRB1-B, wherein CRB1-A is operably linked to a ubiquitous control or regulatory element and CRB1-B is operably linked to a tissue-specific or cell type-specific control or regulatory element. In some embodiments, the composition comprises a single viral vector encoding CRB1-A and CRB1-B, wherein both CRB1-A and CRB1-B are operably linked to a tissue-specific or cell type-specific control or regulatory element. In some embodiments, the single viral vector is derived from a lentivirus.

In some embodiments, the composition comprises two viral vectors, collectively encoding CRB1-A and CRB1-B, wherein the first viral vector encodes CRB1-A and the second viral vector encodes CRB1-B, and one or both of CRB1-A and CRB1-B are operably linked to a tissue-specific or cell type-specific control or regulatory element. In some embodiments, the composition comprises two viral vectors, wherein the first viral vector encodes CRB1-A operably linked to a ubiquitous control or regulatory element and the second viral vector encodes CRB1-B operably linked to a tissue-specific or cell type-specific control or regulatory element. In some embodiments, the composition comprises two viral vectors, wherein the first viral vector encodes CRB1-A and the second viral vector encodes CRB1-B, and both of CRB1-A and CRB1-B are operably linked to a tissue-specific or cell type-specific control or regulatory element. In some embodiments, at least one or both of the two viral vectors are derived from an AAV virus.

In some embodiments, the CRB1-A transgene is operably linked to a Müller glial regulatory element (e.g., a regulatory element (e.g., a promoter) that confers selective or predominantly selective expression of the operably linked gene in a Müller glial cell). In select embodiments, the Müller glial regulatory element comprises a promoter selected from: RLBP1 (Retinaldehyde Binding Protein 1), GFAP (Glial fibrillary acidic protein), GfaABC1D (a truncated GFAP promoter), and synthetic promoters ProB2 and PROC17.

In some embodiments, the composition comprises a single viral vector (e.g., lentivirus vector) comprising a CRB1-A transgene operably linked to a Müller glial regulatory element. In some embodiments, the composition comprises two viral vectors, wherein one of the viral vectors comprises a CRB1-A transgene operably linked to a Müller glial regulatory element.

In some embodiments, the CRB1-B transgene is operably linked to a photoreceptor regulatory element (e.g., a regulatory element that confers selective or predominantly selective expression of the operably linked gene in a photoreceptor cell). Suitable photoreceptor-specific regulatory elements include, but are not limited to, a rhodopsin promoter; a rhodopsin kinase promoter; a beta phosphodiesterase gene promoter; a retinitis pigmentosa gene promoter; an interphotoreceptor retinoid-binding protein (IRBP) gene enhancer; an IRBP gene promoter, an opsin gene promoter, a retinoschisin gene promoter, a CRX homeodomain protein gene promoter, a guanine nucleotide binding protein alpha transducing activity polypeptide 1 (GNAT1) gene promoter, a neural retina-specific leucine zipper protein (NRL) gene promoter, human cone arrestin (hCAR) promoter, and the PR2.1, PR1.7, PR1.5, and PR1.1 promoters. In select embodiments, the photoreceptor-specific regulatory element comprises a promoter selected from: interphotoreceptor retinoid-binding protein (IRBP), cone arrestin (CAR), rhodopsin (RHO), PR1.7 (a truncated version of version of the L-opsin promoter), synthetic promoters: ProA1, ProA6, ProC1, ProA14, and ProA36, and G protein-coupled receptor kinase 1 (GRK1).

In some embodiments, the composition comprises a single viral vector (e.g., lentivirus vector) comprising a CRB1-B transgene operably linked to a photoreceptor regulatory element. In some embodiments, the composition comprises two viral vectors, wherein one of the viral vectors comprises a CRB1-B transgene operably linked to a photoreceptor regulatory element.

### Exemplary CRB1 Isoform Dual Expression Vector

The current disclosure provides for compositions containing a recombinant lentivirus containing a nucleic acid that encodes CRB1-A and CRB1-B on the same dual expression vector under control of separate promoters that induce expression of the transgenes in the proper cells, e.g., CRB1-A in a Müller glial cells and CRB1-B in photoreceptor cells.

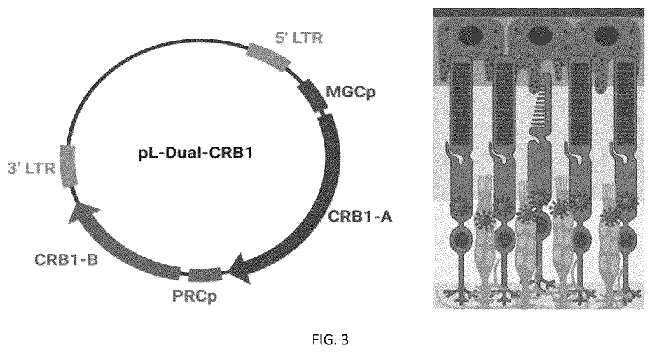

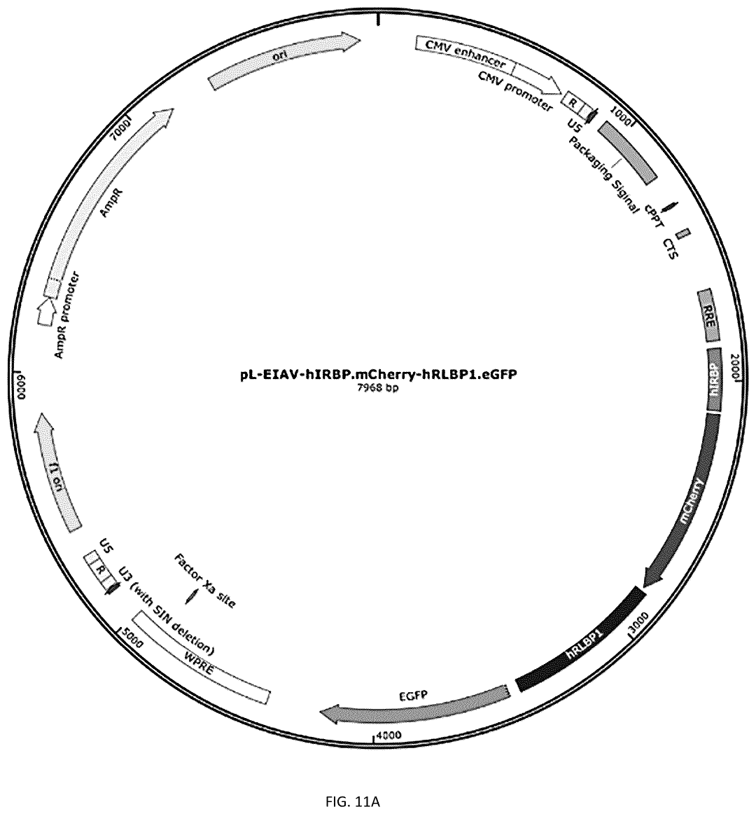

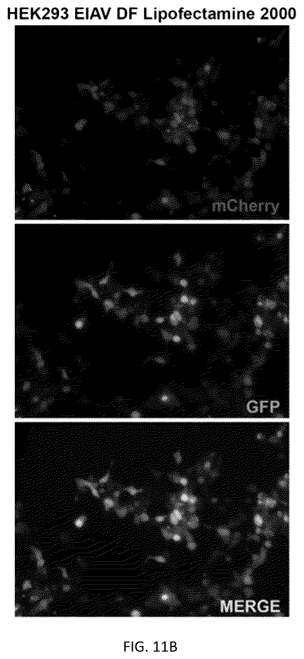

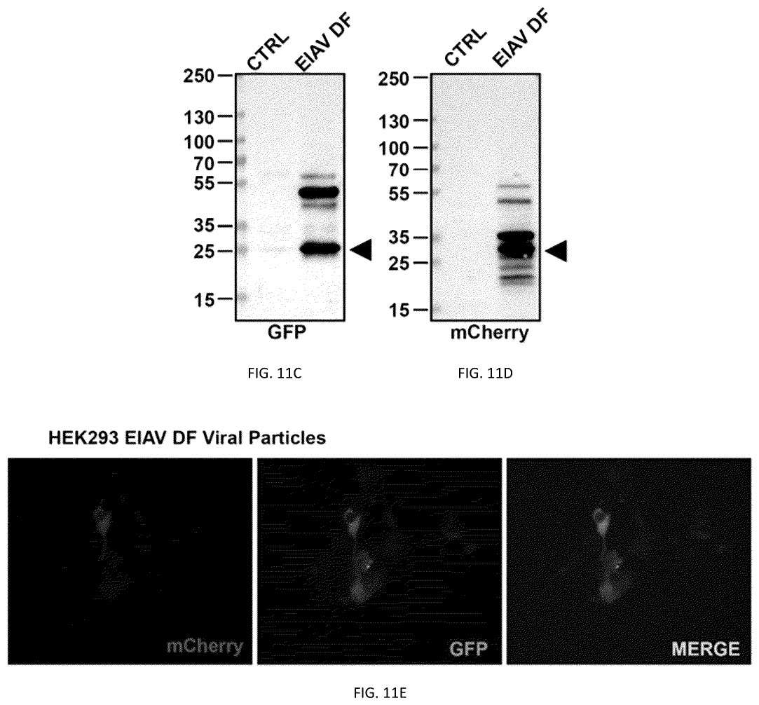

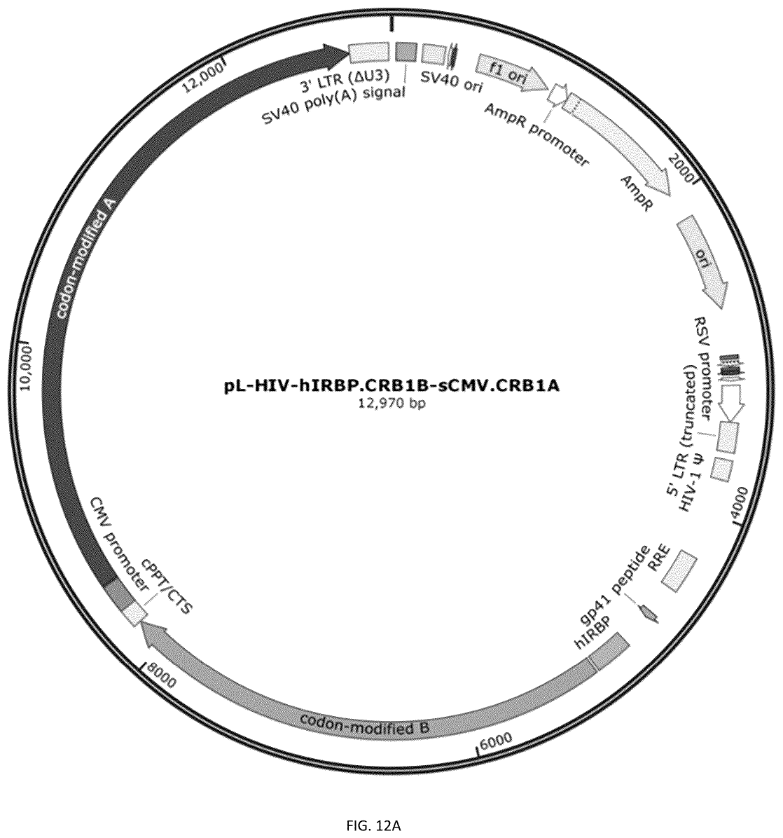

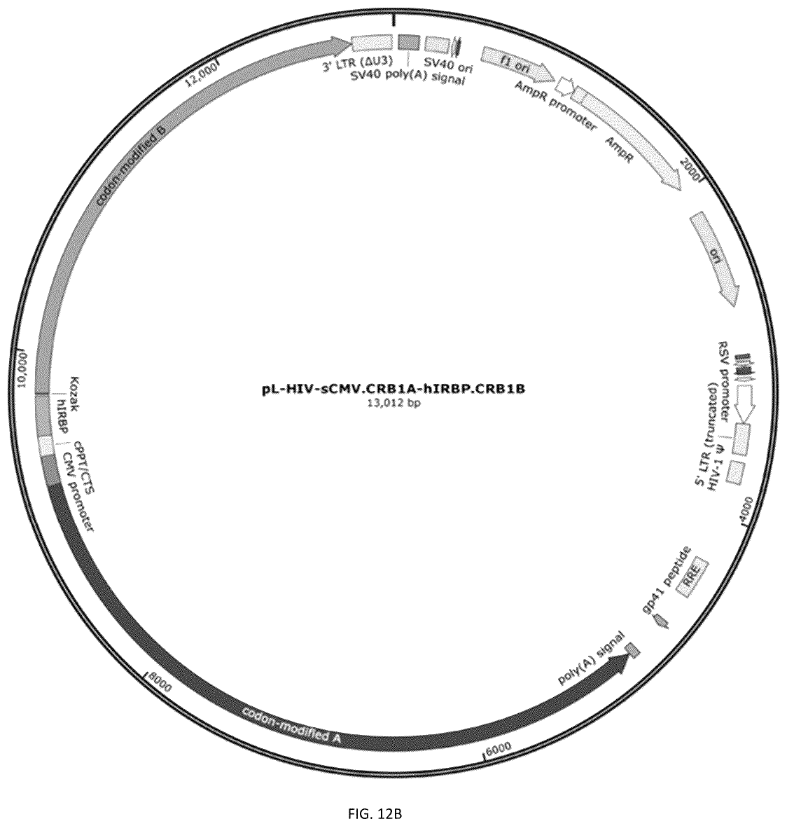

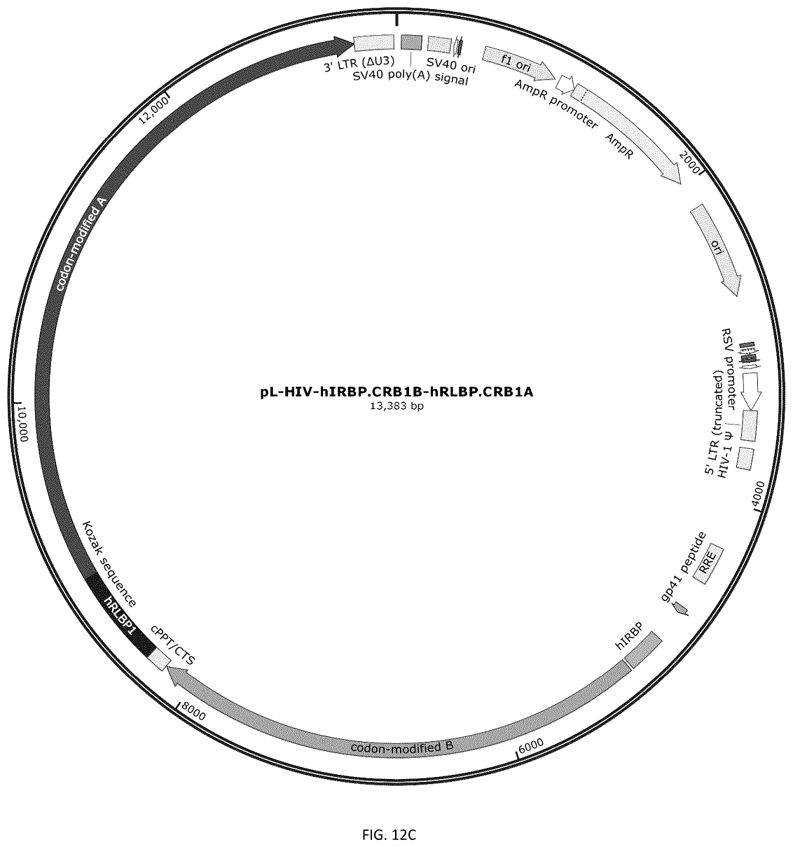

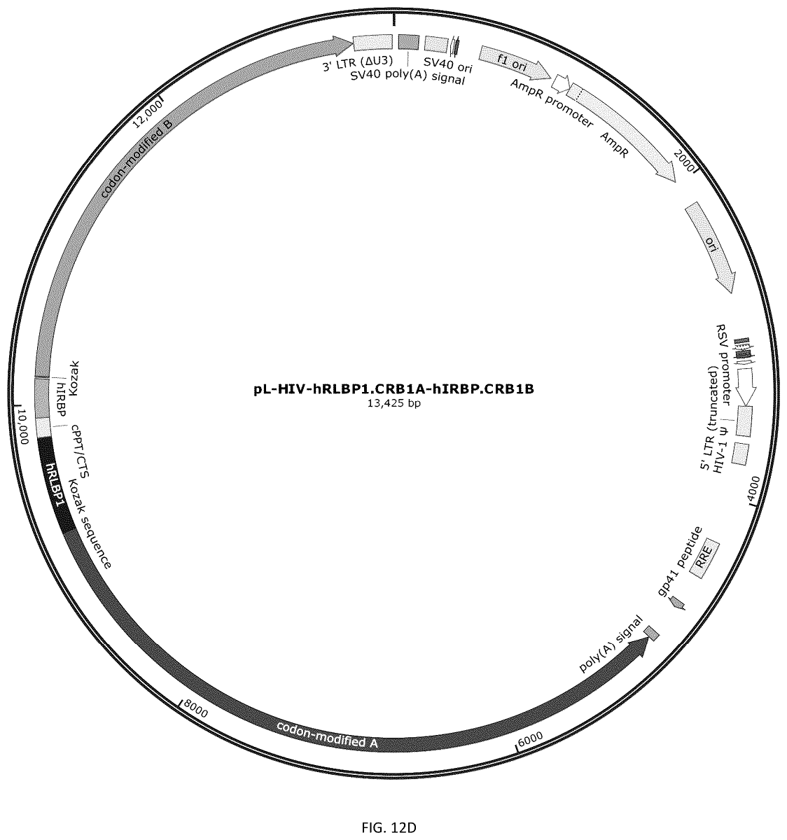

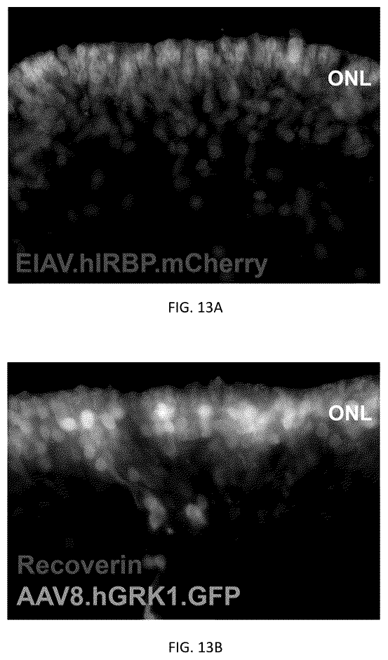

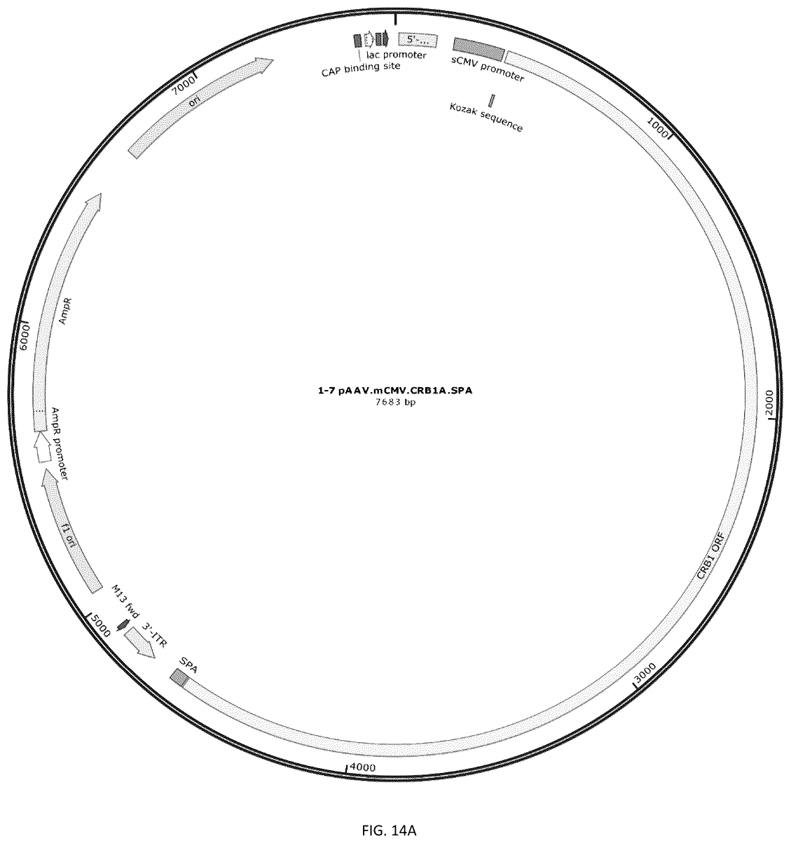

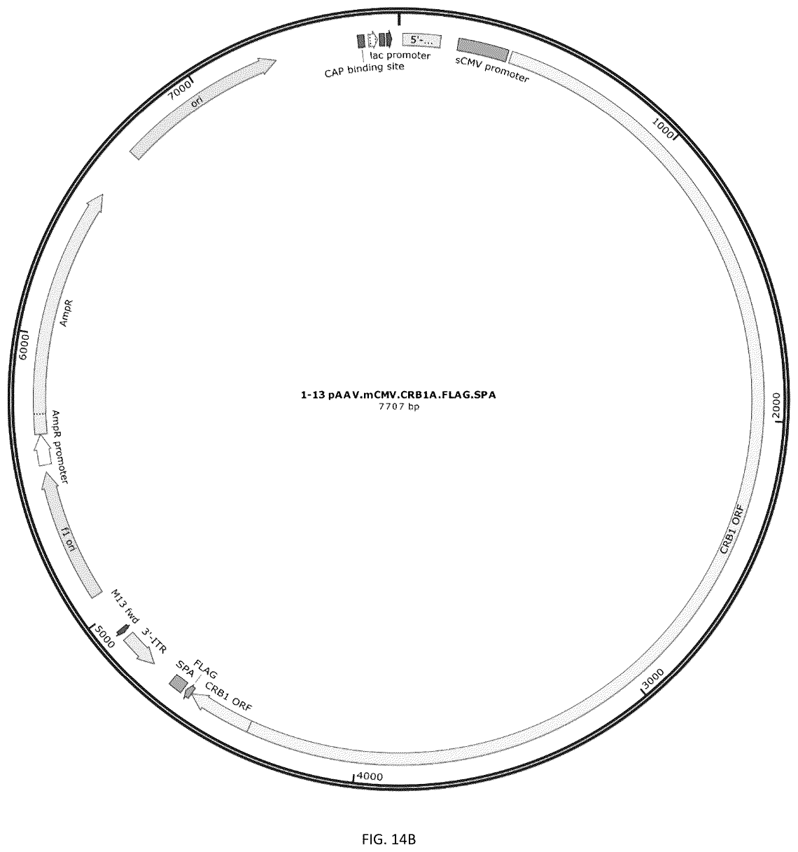

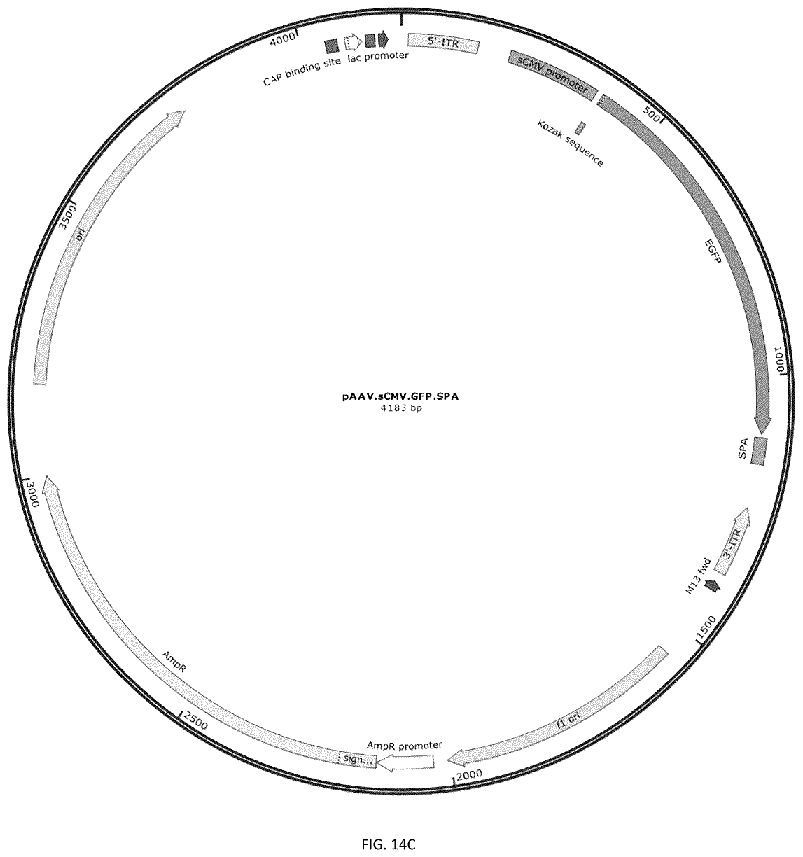

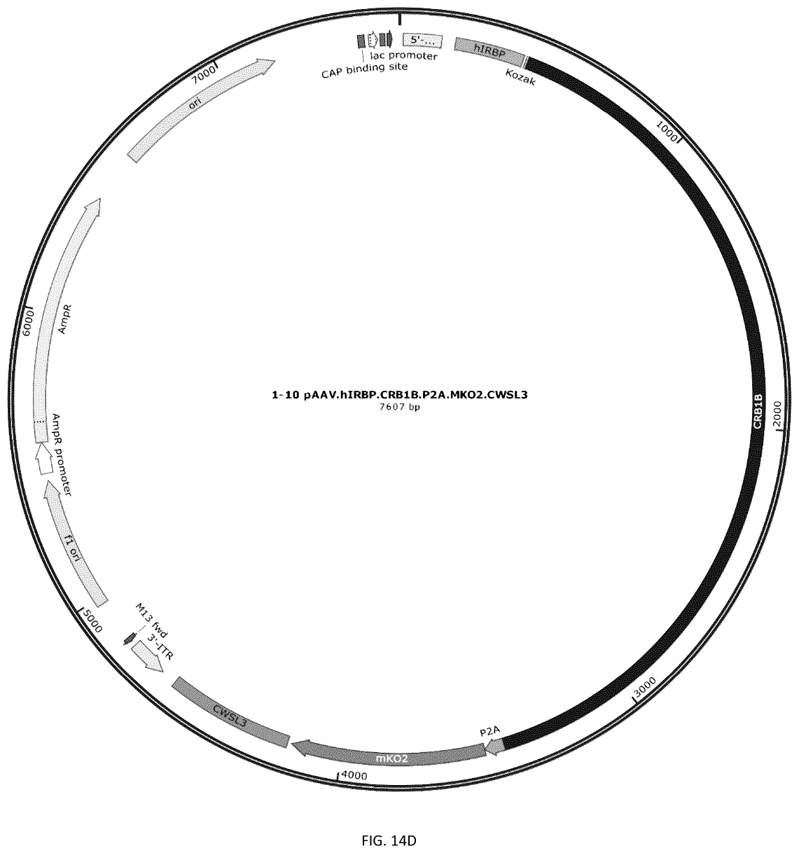

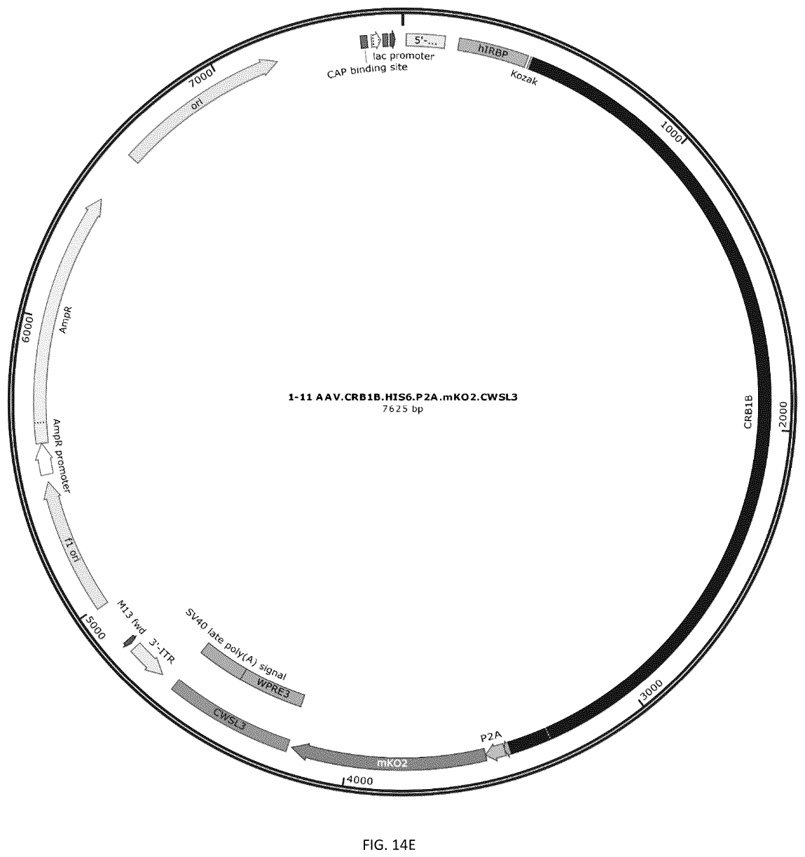

An exemplary CRB1 Isoform Dual Expression Vector is shown in FIG. 3. Exemplary dual expression vectors suitable for use herein are also shown in FIGS. 11A, 12A, and 12B.

In some embodiments, a dual expression vector comprises, from 5′ to 3′, a sequence encoding a first CRB1 isoform and a second CRB1 isoform. In some embodiments, the first and second CRB1 isoforms are independently selected from CRB1-A and CRB1-B. In some embodiments, a dual expression vector comprises, from 5′ to 3′, a sequence encoding a CRB1-A transgene and a sequence encoding a CRB1-B transgene. In some embodiments, a dual expression vector comprises, from 5′ to 3′, a sequence encoding a CRB1-B transgene and a sequence encoding a CRB1-A transgene.

In some embodiments, a dual expression vector comprises, from 5′ to 3′, a first promoter, a sequence encoding a CRB1-A transgene, a second promoter, and a sequence encoding a CRB1-B transgene. In some embodiments, a dual expression vector comprises, from to 3′, a first promoter, a sequence encoding a CRB1-B transgene, a second promoter, and a sequence encoding a CRB1-A transgene. In some embodiments, the CRB1-A transgene is a human CRB1-A transgene. In some embodiments, the CRB1-B transgene is a human transgene. In some embodiments, the CRB1-A transgene is a human CRB1-A transgene and the CRB1-B transgene is a human transgene.

In some embodiments, the CRB1-A transgene comprises one or more amino acid mutations relative to the human CRB1-A sequence. In some embodiments, the CRB1-B transgene comprises one or more amino acid mutations relative to the human CRB1-B sequence. In some embodiments, both the CRB1-A transgene and the CRB1-B transgene comprise one or more amino acid mutations relative to their respective human sequences.

In some embodiments, a dual expression vector further comprises an enhancer, such as the CMV enhancer.

In some embodiments, a dual expression vector further comprises a sequence encoding a reporter, such as a sequence encoding GFP or mCherry.

In some embodiments, a dual expression vector further comprises sequences which facilitate packaging into a viral vector, e.g., lentiviral long terminal repeats (LTRs.)

In some embodiments, a dual expression vector comprises, from 5′ to 3′, a first lentiviral LTR, a sequence encoding a CRB1-A transgene, a sequence encoding a CRB1-B transgene, and a second lentiviral LTR. In some embodiments, a dual expression vector comprises, from 5′ to 3′, a sequence encoding a first lentiviral LTR, a CRB1-B transgene, a sequence encoding a CRB1-A transgene, and a second lentiviral LTR. In some embodiments, a dual expression vector comprises, from 5′ to 3′, a first lentiviral LTR, a first promoter, a sequence encoding a CRB1-A transgene, a second promoter, a sequence encoding a CRB1-B transgene, and a second lentiviral LTR. In some embodiments, a dual expression vector comprises, from 5′ to 3′, a first lentiviral LTR, a first promoter, a sequence encoding a CRB1-B transgene, a second promoter, a sequence encoding a CRB1-A transgene, and a second lentiviral LTR.

In some embodiments, a dual expression vector, as described herein, does not include any heterologous control or regulatory sequences, as described above, between the two CRB1 transgenes. For example, in some embodiments, a dual expression vector comprises, from 5′ to 3′, a sequence encoding a first CRB1 isoform operably linked to a promoter and a second CRB1 isoform operably linked to a promoter without any heterologous control or regulatory sequences (e.g., polyadenylation sequence) between the two transgenes, e.g., 3′ of the sequence encoding a first CRB1 isoform and 5′ of the promoter of a second CRB1 isoform. Exclusion of heterologous control or regulatory sequences between the two transgenes may confer increased expression of one or both of the transgenes compared to a vector including heterologous control or regulatory sequences between the two transgenes. Alternatively or additionally, exclusion of heterologous control or regulatory sequences between the two transgenes may decrease gene truncation events of one or both of the transgenes.

In some embodiments, a dual expression vector, as described herein, includes endogenous, or viral vector derived, control, regulatory, or packaging sequences between the two CRB1 transgenes. In some embodiments, a dual expression vector comprises, from 5′ to 3′, a sequence encoding a first CRB1 isoform operably linked to a promoter and a second CRB1 isoform operably linked to a promoter, separated by endogenous, or viral vector derived, control, regulatory, or packaging sequences. In some embodiments, the endogenous, or viral vector derived, transcription or translational regulatory or packaging sequences include a sequence comprising a central polypurine tract (cPPT) with downstream central termination sequence (CTS).

Methods of Treating, Preventing, and/or Curing Disease or Disorder Characterized by CRB1 Mutations

Patients who would benefit from the administration of the described gene therapy include those diagnosed with a disease or disorder characterized by CRB1 mutations including but not limited to autosomal recessive retinitis pigmentosa (RP) and Leber congenital amaurosis (LCA).

In some embodiments, the present disclosure provides methods of treating, preventing, curing, and/or reducing the severity or extent of a disease or disorder characterized by CRB1 mutations including but not limited to autosomal recessive retinitis pigmentosa (RP) and Leber congenital amaurosis (LCA) by administering to a subject in need thereof a therapeutically effective amount of a composition, such as a viral vector or vectors, comprising a nucleic acid encoding more than one isoform of CRB1, such as CRB1-A and CRB1-B.

In some embodiments, the methods comprise administering to a subject in need thereof a dual expression vector, as described herein.

In some embodiments, the methods comprise administering to a subject in need thereof a composition comprising more than one vector, each vector comprising at least one isoform of CRB1. For example, the composition may comprise a first vector encoding a first CRB1 isoform and a second vector encoding a second CRB1 isoform. In some embodiments, the methods comprise administering to a subject in need thereof a composition comprising a first vector encoding a CRB1-A transgene and a second vector encoding a CRB1-B transgene, as described herein.

In some embodiments, the composition (e.g., viral vector or vectors) comprising a nucleic acid encoding more than one isoform of CRB1, such as CRB1-A and CRB1-B is administered as soon as the disease or disorder is characterized by CRB1 mutations. The disease or disorder may include, but is not limited to, autosomal recessive retinitis pigmentosa (RP) and Leber congenital amaurosis (LCA).

In some embodiments, the present disclosure provides methods of treating, preventing, curing, and/or reducing the severity or extent of a disease or disorder characterized by CRB1 mutations including but not limited to autosomal recessive retinitis pigmentosa (RP) and Leber congenital amaurosis (LCA) by administering to a subject in need thereof a therapeutically effective amount of a composition, such as a viral vector, comprising a nucleic acid encoding an isoform of CRB1, such as CRB1-A or CRB1-B, configured to allow expression of the CRB1 isoform in more than one retinal cell type (e.g., Müller glial cells and photoreceptor cells).

In some embodiments, the composition comprises a viral vector encoding CRB1-A. In some embodiments, the composition comprises a viral vector encoding CRB1-B. In some embodiments, the transgenes and vector are configured to allow expression in Müller glial cells and photoreceptor cells.

### Routes of Administration and Dosing

The current disclosure provides for compositions and vectors for use in methods of treating, preventing, and/or curing a disease or disorder characterized by CRB1 mutations including but not limited to autosomal recessive retinitis pigmentosa (RP) and Leber congenital amaurosis (LCA) and/or alleviating in a subject at least one of the symptoms associated with a disease or disorder characterized by CRB1 mutations including but not limited to autosomal recessive retinitis pigmentosa (RP) and Leber congenital amaurosis (LCA). In some embodiments, methods involve administration of the compositions and vectors, in a pharmaceutically-acceptable carrier to the subject in an amount and for a period of time sufficient to treat, prevent and/or cure the characterized by CRB1 mutations including but not limited to autosomal recessive retinitis pigmentosa (RP) and Leber congenital amaurosis (LCA).

In certain embodiments, the route of administration is subretinal injection or intravitreal injection.

The vector can be formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween-20.

In another embodiment, the pharmaceutically acceptable carrier comprises a surfactant, such as perfluorooctane (Perfluoron liquid). In certain embodiments, the pharmaceutical composition described above is administered to the subject by subretinal injection. In other embodiments, the pharmaceutical composition is administered by intravitreal injection.

Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired.

Suitable carriers may be readily selected by one of skill in the art in view of the indication. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions may contain, in addition to the vector and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1% or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active vector in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The present invention provides stable pharmaceutical compositions comprising virions. The compositions remain stable and active even when subjected to freeze/thaw cycling and when stored in containers made of various materials, including glass.

Appropriate doses will depend on the subject being treated (e.g., human or nonhuman primate or other mammal), age and general condition of the subject to be treated, the severity of the condition being treated, the mode of administration of the vector, among other factors. An appropriate effective amount can be readily determined by one of skill in the art.

The dose of vector required to achieve a desired effect or “therapeutic effect,” e.g., the units of dose in vector genomes/per kilogram of body weight (vg/kg), will vary based on several factors including, but not limited to: the route of administration; the level of gene or RNA expression required to achieve a therapeutic effect; the specific disease or disorder being treated; and the stability of the gene or RNA product. One of skill in the art can readily determine a dose range to treat a subject having a particular disease or disorder based on the aforementioned factors, as well as other factors that are well known in the art. An effective amount is generally in the range of from about 10 μl to about 100 ml of solution containing from about 10⁹ to 10¹⁶ genome copies per subject. Other volumes of solution may be used. The volume used will typically depend, among other things, on the size of the subject, the dose, and the route of administration.

Dosage treatment may be a single dose schedule or a multiple dose schedule to ultimately deliver the amount specified above. Moreover, the subject may be administered as many doses as appropriate. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, the vector will be present in the subject compositions in an amount sufficient to provide a therapeutic effect when given in one or more doses.

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Identification of Isoform Specific Distribution of CRB1 Isoforms

It was found that approximately 70% of novel CRB1 patient mutations affect both CRB1-A and CRB1-B (FIG. 1).

Using in situ hybridization (Basescope), isoform specific distribution of CRB1-A, B and C was found in the human donor retina and human retinal organoids (ROs) (FIG. 2).

A probe which targeted all three isoforms at exon 6 (PAN-CRB1) localized in both the ONL and the INL as well as the inner segments of the photoreceptor. The CRB1-A probe targeted exon 12 (FIGS. 2C and 2D). The signal for CRB1-A was strongly expressed in the INL, particularly in the maturating retinal organoids. However, a significant proportion of CRB1-A transcript was still found localized to the ONL.

The CRB1-B probe targeted its unique exon 1. The signal for CRB1-B was primarily expressed in the ONL and inner segments and to a lesser extent the INL.

The CRB1-C probe targeted its unique exon 6. The signal was localized in both the ONL and INL and to a lesser extent than CRB1-B in the inner segments.

The expression patterns of CRB1-B and CRB1-C appear to more evenly distributed between the ONL and INL of maturating retinal organoids.

Thus, it was determined that CRB1-A is predominantly expressed in Müller glial cells and CRB1-B in photoreceptor cells.

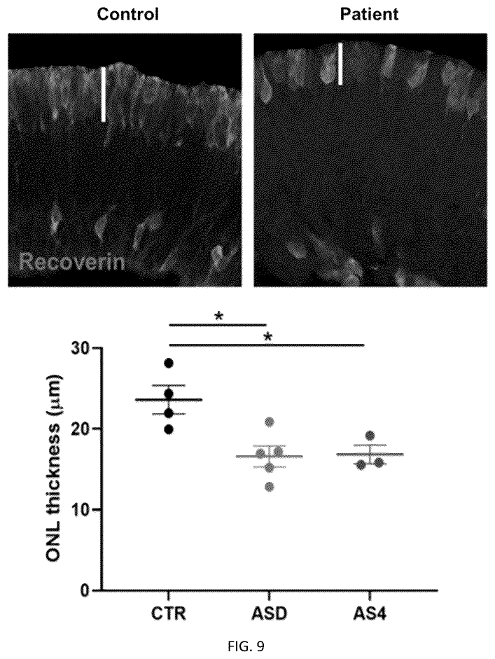

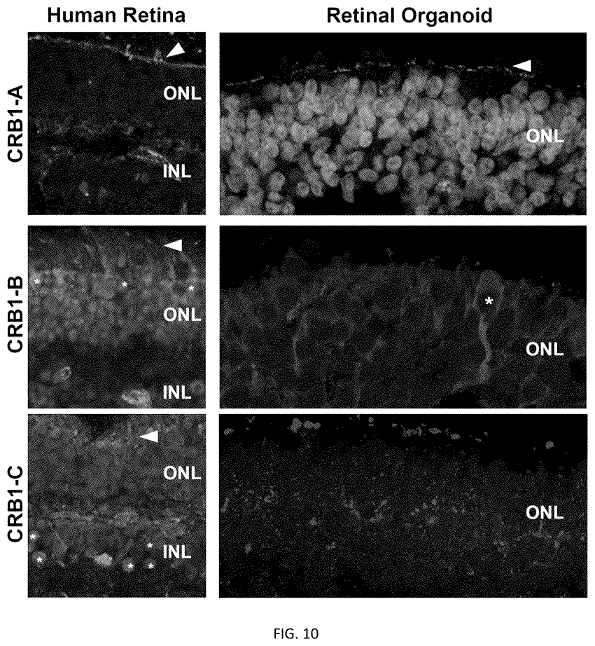

Additionally, the phenotypes observed in CRB1 LCA ROs are similar to those exhibited in patients suffering from LCA (FIG. 9).

### Example 2—CRB1 Isoform Dual Expression Vector Construction

Using the information in Example 1 that CRB1-A is predominantly expressed in Müller glial cells and CR1-B in photoreceptor cells, a dual expression vector was designed which mediates concomitant CRB1-A and CRB1-B expression to their predominately expressed cell types as found in wild-type retina.

CRB1-A which is predominantly expressed in Müller glial cells is linked to promoters including but not limited to a RLBP1, GFAP, and PROC17. CRB1-B which is predominantly expressed in photoreceptor cells is linked to promoters including but not limited to IRBP and GRK1. See FIG. 3 for schematic of exemplary strategy.

An alternative approach targets both isoforms concomitantly to both cell types using ubiquitous promoters such as CMV, EF1, CAG, and SFFV. Both isoforms along with cell type specific or ubiquitous promoters along with post-transcriptional regulatory elements (e.g., WPRE) and polyadenylation sequences for efficient pre-mRNA processing (e.g., SV40) is placed in HIV and EIAV lentiviral based vectors for testing.

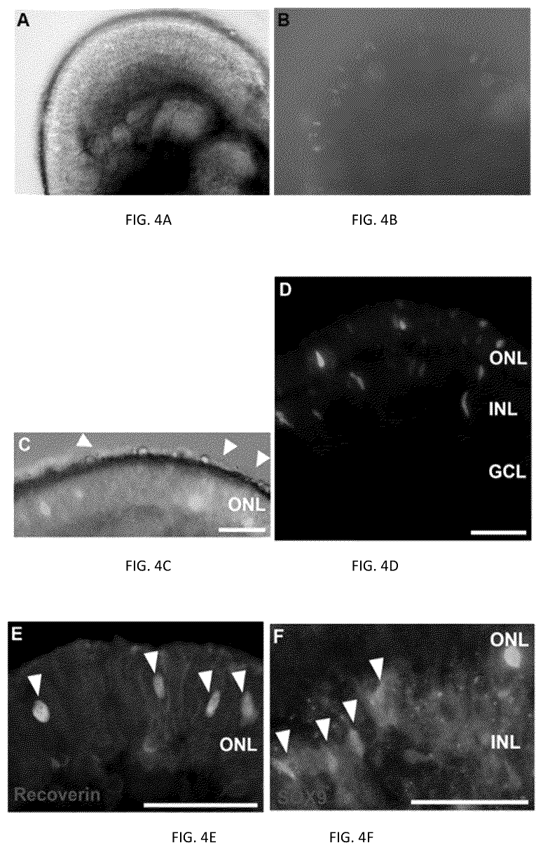

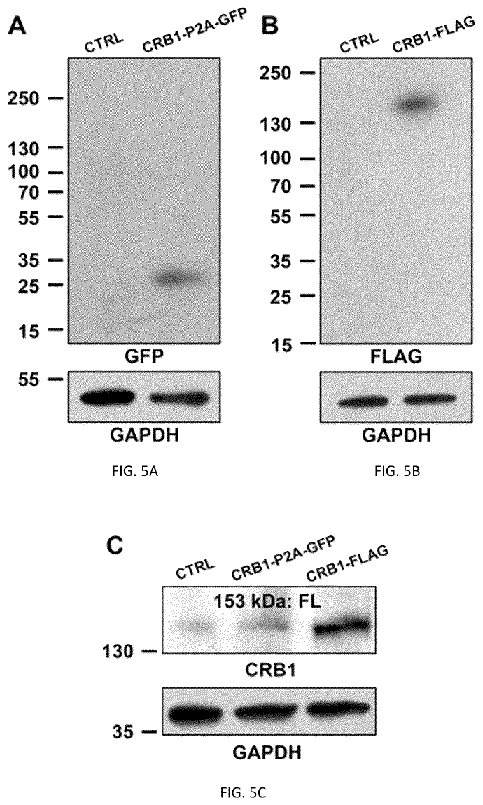

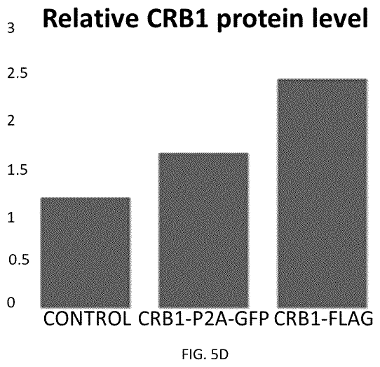

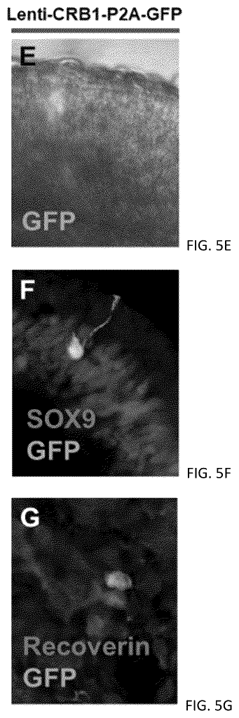

Previous work has produced multiple CRB1-A lentiviral based vectors (FIGS. 4 and 5), which are adapted for the production of CRB1 Isoform Dual Expression Vector. Both PRCs and MGCs in ROs were successfully targeted using viral vectors with ubiquitous and cell-type specific promoters (FIG. 4).

### Example 3—Retinal Organoids

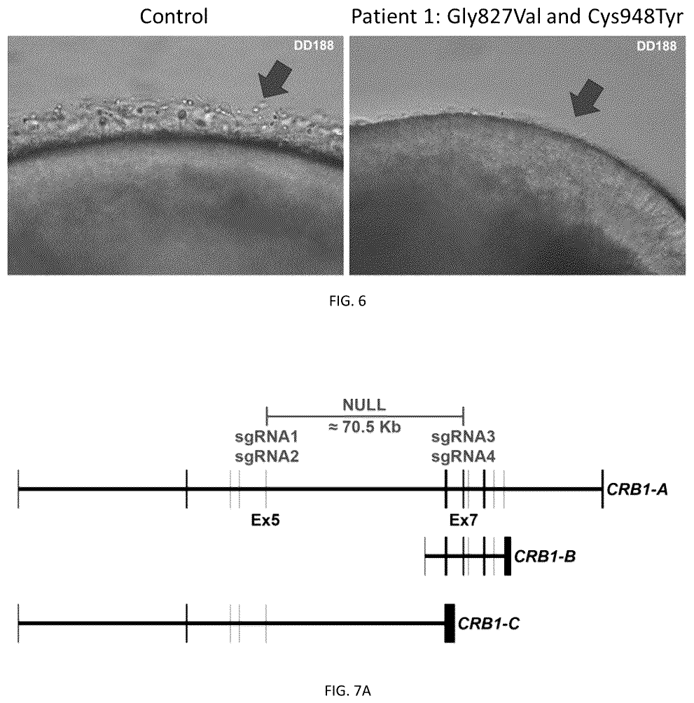

Three CRB1 patients, all having mutations that affect CRB1-A and CRB1-B (Table 1 and 2), have been recruited. Induced pluripotent stem cells and retinal organoids have been derived from P001 and P002 (Table 1). CRB1 patient derived retinal organoids have a phenotype, the failure in biosynthesis of photoreceptor outer segments, which can be used as an outcome measurement for therapeutic efficacy. See FIG. 6. Table 3 highlights the 10 Most Frequent CRB1 pathogenic Variants in the Leiden Open Variation Database.

TABLE 1

| Patient # | CRB1 Mutation |
|---|---|
| P001 | compound heterozygote c.2843G>A:p.(Cys948Tyr) and c.2480G>T:p.(Gly827Val) |
| P002 | homozygous c.3307G>A p. (Gly1103Arg) |
| P003 | compound heterozygote c.2245_2247delTCA:p.(Ser749del) and |
| | c.2843G>A:p.(Cys948Tyr) |

TABLE 2

| | CRB1 Isoforms |
|---|---|
| CRB1 Mutation | affected |
| c.2245_2247delTCA:p.(Ser749del) | A and B |
| c.2480G>T:p.(Gly827Val) | A and B |
| c.2843G>A:p.(Cys948Tyr) | A and B |
| c.3307G>A p. (Gly1103Arg) | A and B |

TABLE 3

| | | | | | Proportion of |
|---|---|---|---|---|---|
| | | cDNA Change | Protein Change | Alleles (n) | pathogenic alleles (%) |
| LOVD | 1 | c.2843G>A | p.(Cys948Tyr) | 172 | 13.66 |
| | 2 | c.2401A>T | p.(Lys801*) | 50 | 3.97 |
| | 3 | c.2234C>T | p.(Thr745Met) | 47 | 3.73 |
| | 4 | c.2290C>T | p.(Arg764Cys) | 39 | 3.10 |
| | 5 | c.2688T>A | p.(Cys896*) | 29 | 2.30 |
| | 6 | c.613_619del | p.(Ile205Aspfs*13) | 27 | 2.14 |
| | 7 | c.498_506del | p.(Ile167_Gly169del) | 22 | 1.75 |
| | 8 | c.3307G>A | p.(Gly1103Arg) | 20 | 1.59 |
| | 9 | c.4121_4130del | p.(Ala1374Glufs*20) | 17 | 1.35 |
| | 10 | c.1148G>A | p.(Cys383Tyr) | 17 | 1.35 |

| | | |
|---|---|---|
| Total | 440 | 34.95 |

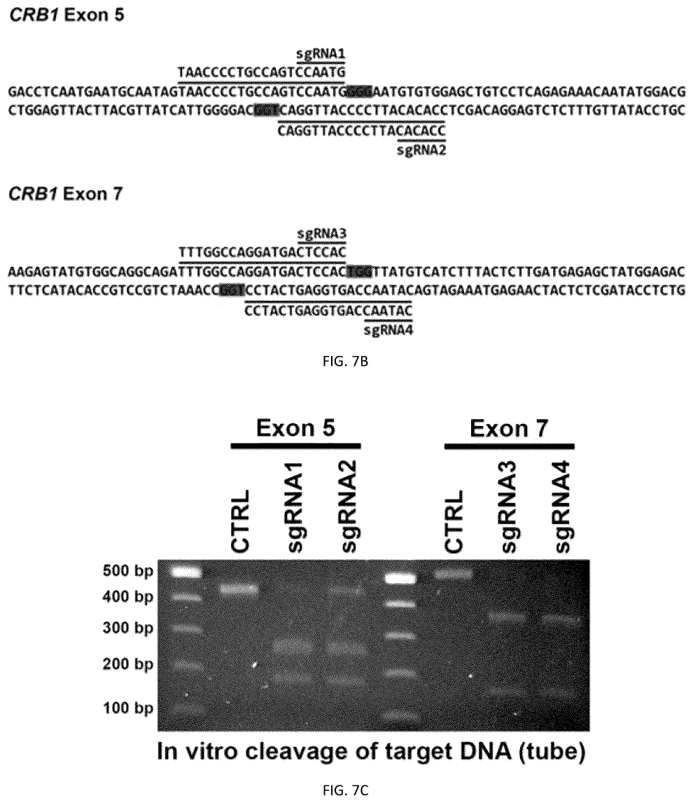

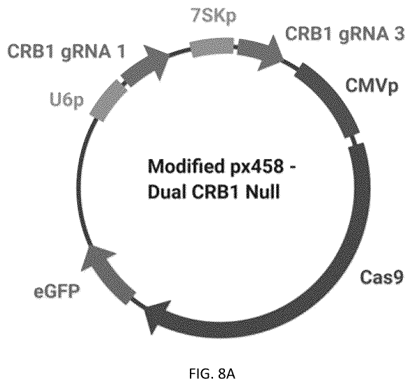

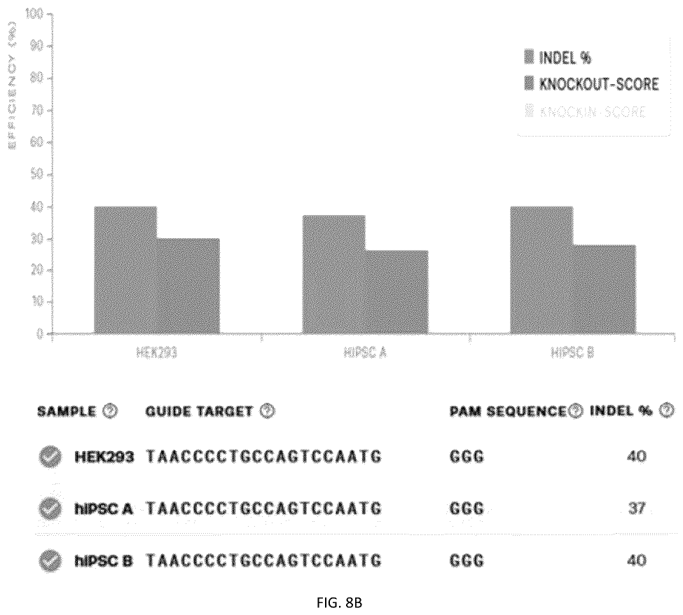

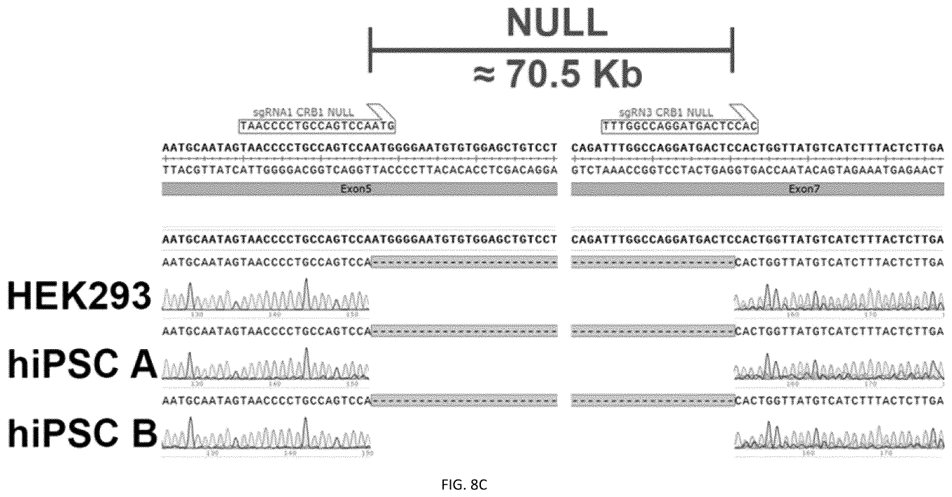

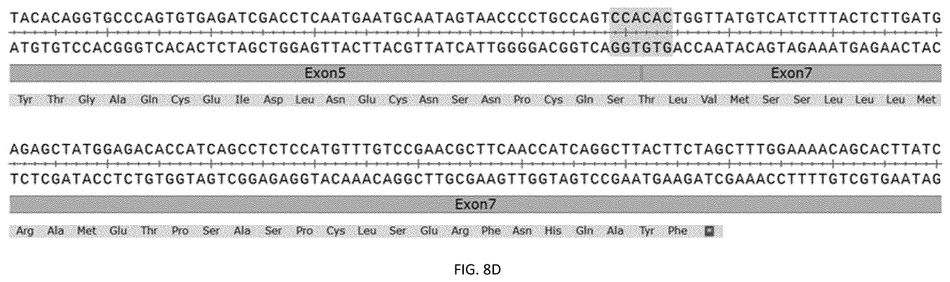

As an additional strategy, CRB1 null iPSCs are generated for their derivation to CRB1 Null retinal organoids. These can be utilized as optimal model for testing CRB1 gene augmentation. See FIGS. 7 and 8.

Previous studies have demonstrated that ROs can be generated from CRB1 Retinitis pigmentosa patient derived iPSCs and that these ROs exhibit a morphological phenotype of outer limiting membrane disruptions (OLM) and ectopic photoreceptor localization. To test this hypothesis, patient iPSCs were used to generate CRB1 LCA retinal organoids (ROs). Initial data suggest that the phenotypes observed in CRB1 RP and LCA organoids are similar to those exhibited in patients and that the therapeutic agents can successfully target ROs. These data align with the hypothesis that patient iPSC ROs will be clinically-relevant recipients of therapeutic agents. The rationale for this project is that iPSC ROs recapitulate both human retinal development and the naive protein expression patterns of CRB1 and also phenocopy retinal disease pathogenesis, rendering them an excellent tool for understanding disease mechanisms and a platform for testing therapeutic strategies.

### Example 4— Results Using CRB1 Isoform Dual Expression Vector Versus Single Expression Vectors

The following vectors are administered to the retinal organoids generated from patients with either RP or LCA described in Example 3:

A CRB1 dual expression vector containing CRB1-A transgene under control of a promoter which induces expression in Müller glial cells and CRB1-B transgene under the control of a promoter which induces expression in photoreceptor cells as described above;

A CRB1 dual expression vector containing CRB1-A transgene and CRB1-B transgene under the control of a ubiquitous promoter as described above;

A vector containing a CRB1-A transgene; and

A vector containing a CRB1-B transgene.

The same vectors are also administered to rat models of disease both via subretinal injection or intravitreal injection.

Both the ROs and the rats are assessed for the following:
Improvement in morphological phenotype including failure of biosynthesis of photoreceptor outer segments, retinal thickening, abnormal lamination, outer limiting membrane disruption and adherens junctions (AJs) instability;
Improvement in functional phenotype;
Survival;
Expression of the wild-type isoform; and
Localization.

The dual expression vectors show greater improvement in morphology, function, and survival than either single expression vector. Moreover, as expected only the ROs or rats that received the dual expression vector expressed both of the wild-type isoforms. The CRB1 isoforms are expressed in the correct cells when the dual expression vector is administered.

As an additional model, a mouse model of spontaneous retinal vascularization JR5558 B6. Cg-Crb1^{rd8} Jak3^{mIJ}/Boc mice (Chang, et al., Invest Ophthalmol Vis Sci. 2018; 59:5127-5139, incorporated herein by reference in its entirety), which shows diminished ERG from 2 months of age, are utilized in a similar manner to the rat model described above.

### SEQUENCES

| SEQ | | |
|---|---|---|
| ID NO | Description | Sequence |
| 1 | sgRNA1 | TAACCCCTGCCAGTCCAATG |
| 2 | sgRNA2 | CCACACATTCCCCATTGGAC |
| 3 | sgRNA3 | TTTGGCCAGGATGACTCCAC |
| 4 | sgRNA4 | CATAACCAGTGGAGTCATCC |
| 5 | CRB1 | GACCTCAATGAATGCAATAGTAACCCCTGCCAGTCCA |
| | Exon 5 | ATGGGGAATGTGTGGAGCTGTCCTCAGAGAAACAAT |
| | | ATGGACG |
| 6 | CRB1 | AAGAGTATGTGGCAGGCAGATTTGGCCAGGATGACT |
| | Exon 7 | CCACTGGTTATGTCATCTTTACTCTTGATGAGAGCTA |
| | | TGGAGAC |
| 7 | Combined | TACACAGGTGCCCAGTGTGAGATCGACCTCAATGAA |
| | CRB1 Exon | TGCAATAGTAACCCCTGCCAGTCCACACTGGTTATGT |
| | 5/7 | CATCTTTACTCTTGATGAGAGCTATGGAGACACCATC |
| | | AGCCTCTCCATGTTTGTCCGAACGCTTCAACCATCAG |
| | | GCTTACTTCTAGCTTTGGAAAACAGCACTTATC |
| 8 | Human | MALKNINYLLIFYLSFSLLIYIKNSFCNKNNTRCLSNSCQ |
| | CRB1-A | NNSTCKDFSKDNDCSCSDTANNLDKDCDNMKDPCFSN |
| | | PCQGSATCVNTPGERSFLCKCPPGYSGTICETTIGSCGK |
| | | NSCQHGGICHQDPIYPVCICPAGYAGRFCEIDHDECASS |
| | | PCQNGAVCQDGIDGYSCFCVPGYQGRHCDLEVDECAS |
| | | DPCKNEATCLNEIGRYTCICPHNYSGVNCELEIDECWSQ |
| | | PCLNGATCQDALGAYFCDCAPGFLGDHCELNTDECAS |
| | | QPCLHGGLCVDGENRYSCNCTGSGFTGTHCETLMPLC |
| | | WSKPCHNNATCEDSVDNYTCHCWPGYTGAQCEIDLNE |
| | | CNSNPCQSNGECVELSSEKQYGRITGLPSSFSYHEASGY |
| | | VCICQPGFTGIHCEEDVNECSSNPCQNGGTCENLPGNYT |
| | | CHCPFDNLSRTFYGGRDCSDILLGCTHQQCLNNGTCIPH |
| | | FQDGQHGFSCLCPSGYTGSLCEIATTLSFEGDGFLWVKS |
| | | GSVTTKGSVCNIALRFQTVQPMALLLFRSNRDVFVKLE |
| | | LLSGYIHLSIQVNNQSKVLLFISHNTSDGEWHFVEVIFAE |
| | | AVTLTLIDDSCKEKCIAKAPTPLESDQSICAFQNSFLGGL |
| | | PVGMTSNGVALLNFYNMPSTPSFVGCLQDIKIDWNHIT |
| | | LENISSGSSLNVKAGCVRKDWCESQPCQSRGRCINLWL |
| | | SYQCDCHRPYEGPNCLREYVAGRFGQDDSTGYVIFTLD |
| | | ESYGDTISLSMFVRTLQPSGLLLALENSTYQYIRVWLER |
| | | GRLAMLTPNSPKLVVKFVLNDGNVHLISLKIKPYKIELY |
| | | QSSQNLGFISASTWKIEKGDVIYIGGLPDKQETELNGGF |
| | | FKGCIQDVRLNNQNLEFFPNPTNNASLNPVLVNVTQGC |
| | | AGDNSCKSNPCHNGGVCHSRWDDFSCSCPALTSGKAC |
| | | EEVQWCGFSPCPHGAQCQPVLOGFECIANAVENGQSGQ |
| | | ILFRSNGNITRELTNITFGFRTRDANVIILHAEKEPEFLNIS |
| | | IQDSRLFFQLQSGNSFYMLSLTSLQSVNDGTWHEVTLS |
| | | MTDPLSQTSRWQMEVDNETPFVTSTIATGSLNFLKDNT |
| | | DIYVGDRAIDNIKGLQGCLSTIEIGGIYLSYFENVHGFIN |
| | | KPQEEQFLKISTNSVVTGCLQLNVCNSNPCLHGGNCEDI |
| | | YSSYHCSCPLGWSGKHCELNIDECFSNPCIHGNCSDRVA |
| | | AYHCTCEPGYTGVNCEVDIDNCQSHQCANGATCISHTN |
| | | GYSCLCFGNFTGKFCRQSRLPSTVCGNEKTNLTCYNGG |
| | | NCTEFQTELKCMCRPGFTGEWCEKDIDECASDPCVNGG |
| | | LCQDLLNKFQCLCDVAFAGERCEVDLADDLISDIFTTIG |
| | | SVTVALLLILLLAIVASVVTSNKRATQGTYSPSRQEKEG |
| | | SRVEMWNLMPPPAMERLI |
| 9 | Human | MFGARTHGFHILMAMLIGIHCEEDVNECSSNPCQNGGT |
| | CRB1-B | CENLPGNYTCHCPFDNLSRTFYGGRDCSDILLGCTHQQ |
| | | CLNNGTCIPHFQDGQHGFSCLCPSGYTGSLCEIATTLSFE |
| | | GDGFLWVKSGSVTTKGSVCNIALRFQTVQPMALLLFRS |
| | | NRDVFVKLELLSGYIHLSIQVNNQSKVLLFISHNTSDGE |
| | | WHFVEVIFAEAVTLTLIDDSCKEKCIAKAPTPLESDQSIC |
| | | AFQNSFLGGLPVGMTSNGVALLNFYNMPSTPSFVGCLQ |
| | | DIKIDWNHITLENISSGSSLNVKAGCVRKDWCESQPCQS |
| | | RGRCINLWLSYQCDCHRPYEGPNCLREYVAGRFGQDD |
| | | STGYVIFTLDESYGDTISLSMFVRTLQPSGLLLALENSTY |
| | | QYIRVWLERGRLAMLTPNSPKLVVKFVLNDGNVHLISL |
| | | KIKPYKIELYQSSQNLGFISASTWKIEKGDVIYIGGLPDK |
| | | QETELNGGFFKGCIQDVRLNNQNLEFFPNPTNNASLNP |
| | | VLVNVTQGCAGDNSCKSNPCHNGGVCHSRWDDFSCSC |
| | | PALTSGKACEEVQWCGFSPCPHGAQCQPVLQGFECIAN |
| | | AVFNGQSGQILFRSNGNITRELTNITFGFRTRDANVIILH |
| | | AEKEPEFLNISIQDSRLFFQLQSGNSFYMLSLTSLQSVND |
| | | GTWHEVTLSMTDPLSQTSRWQMEVDNETPFVTSTIATG |
| | | SLNFLKDNTDIYVGDRAIDNIKGLQGCLSTIEIGGIYLSY |
| | | FENVHGFINKPQEEQFLKISTNSVVTGCLQLNVCNSNPC |
| | | LHGGNCEDIYSSYHCSCPLGWSGKHCELNIDECFSNPCI |
| | | HGNCSDRVAAYHCTCEPGYTGVNCEVDIDNCQSHQCA |
| | | NGATCISHTNGYSCLCFGNFTGKFCRQSRLPSTVCGNEK |
| | | TNLTCYNGGNCTEFQTELKCMCRPGFTGEWCEKDIDEC |
| | | ASDPCVNGGLCQDLLNKFQCLCDVAFAGERCEVDVSS |
| | | LSFYVSLLFWQNLFQLLSYLILRMNDEPVVEWGEQEDY |

### REFERENCES

Choi, et al. (2014). Optimization of AAV expression cassettes to improve packaging capacity and transgene expression in neurons. Mol. Brain. 7, 17.
Den Hollander, et al. (1999). Mutations in a human homologue of Drosophila crumbs cause retinitis pigmentosa (RP12). Nat. Genet. 23, 217-221.
Ray, et al. (2020). Comprehensive identification of mRNA isoforms reveals the diversity of neural cell-surface molecules with roles in retinal development and disease. Nat. Commun. 11:3328.
Tepass, et al. (1990). crumbs encodes an EGF-like protein expressed on apical membranes of Drosophila epithelial cells and required for organization of epithelia. Cell 61, 787-799.

1. A composition comprising transgenes encoding more than one isoform of CRB1, wherein at least one or all of the more than one isoform of CRB1 is operably linked to a tissue-specific or cell type-specific control or regulatory element.

2. The composition of claim 1, wherein each of the more than one isoform of CRB1 is operably linked to a tissue-specific or cell type-specific control or regulatory element.

3. The composition of claim 1, wherein the more than one isoform of CRB1 comprises CRB1-A and CRB1-B.

4. The composition of claim 1, wherein the transgenes encoding more than one isoform of CRB1 are provided on a single vector.

5. The composition of claim 4, wherein the single vector is a viral vector.

6. The composition of claim 5, wherein the viral vector is derived from a virus selected from the group consisting of adeno-associated virus (AAV), adenovirus, lentivirus, retrovirus, poxvirus, baculovirus, herpes simplex virus, vaccinia virus, and a synthetic virus.

7. The composition of claim 5, wherein the viral vector is derived from lentivirus.

8. The composition of claim 1, wherein each of the more than one isoform of CRB1 are operably linked to the same or different promoter.

9. The composition of claim 1, wherein the transgenes encoding more than one isoform of CRB are provided on two or more vectors.

10. The composition of claim 9, wherein the two or more vectors are each individually derived from a virus selected from the group consisting of adeno-associated virus (AAV), adenovirus, lentivirus, retrovirus, poxvirus, baculovirus, herpes simplex virus, vaccinia virus, and a synthetic virus.

11. The composition of claim 10, wherein at least one or both of the two or more vectors are derived from adeno-associated virus.

12. The composition of claim 1, wherein at least one of the more than one isoform of CRB1 is operably linked to constitutive or ubiquitous promoter.

13. The composition of claim 3, wherein the CRB1-A is operably linked to a promoter which induces expression in Müller glial cells.

14. The composition of claim 13, wherein the promoter which induces expression in Müller glial cells is selected from the group consisting of RLBP1, GfaABC1D, GFAP, ProB2 and PROC17.

15. The composition of claim 3, wherein the CRB1-B is operably linked to a promoter which induces expression in photoreceptor cells.

16. The composition of claim 15, wherein the promoter which induces expression in photoreceptor cells is selected from the group consisting of IRBP, CAR, RHO, PR1.7, ProA1, ProA6, ProC1, ProA14, ProA36 and GRK1.

17. A method of treating, preventing, and/or curing a disease or disorder characterized by CRB1 mutations in a subject in need thereof, comprising administering to the subject the composition of claim 1.

18. The method of claim 17, wherein the more than one isoform of CRB1 comprises CRB1-A and CRB1-B.

19. The method of claim 17, wherein the disease or disorder characterized by CRB1 mutations is selected from the group consisting of autosomal recessive retinitis pigmentosa (RP) and Leber congenital amaurosis (LCA).

20. The method of claim 17, wherein the administration is by subretinal injection or intravitreal injection.


SEQUENCE LISTING (9 sequences)

SEQ ID NO: 1 - DNA, 20 bp, Synthetic construct
  source 1..20: mol_type = unassigned DNA
taacccctgccagtccaatg

SEQ ID NO: 2 - DNA, 20 bp, Synthetic construct
  source 1..20: mol_type = unassigned DNA
ccacacattccccattggac

SEQ ID NO: 3 - DNA, 20 bp, Synthetic construct
  source 1..20: mol_type = unassigned DNA
tttggccaggatgactccac

SEQ ID NO: 4 - DNA, 20 bp, Synthetic construct
  source 1..20: mol_type = unassigned DNA
cataaccagtggagtcatcc

SEQ ID NO: 5 - DNA, 80 bp, Homo sapiens
  source 1..80: mol_type = genomic DNA
gacctcaatgaatgcaatagtaacccctgccagtccaatggggaatgtgtggagctgtcctcagagaaacaatatggacg

SEQ ID NO: 6 - DNA, 80 bp, Homo sapiens
  source 1..80: mol_type = genomic DNA
aagagtatgtggcaggcagatttggccaggatgactccactggttatgtcatctttactcttgatgagagctatggagac

SEQ ID NO: 7 - DNA, 180 bp, Synthetic construct
  source 1..180: mol_type = unassigned DNA
tacacaggtgcccagtgtgagatcgacctcaatgaatgcaatagtaacccctgccagtccacactggttatgtcatctttactcttgatgagagctatggagacaccatcagcctctccatgtttgtccgaacgcttcaaccatcaggcttacttctagctttggaaaacagcacttatc

SEQ ID NO: 8 - protein, 1406 aa, Homo sapiens
MALKNINYLLIFYLSFSLLIYIKNSFCNKNNTRCLSNSCQNNSTCKDFSKDNDCSCSDTANNLDKDCDNMKDPCFSNPCQGSATCVNTPGERSFLCKCPPGYSGTICETTIGSCGKNSCQHGGICHQDPIYPVCICPAGYAGRFCEIDHDECASSPCQNGAVCQDGIDGYSCFCVPGYQGRHCDLEVDECASDPCKNEATCLNEIGRYTCICPHNYSGVNCELEIDECWSQPCLNGATCQDALGAYFCDCAPGFLGDHCELNTDECASQPCLHGGLCVDGENRYSCNCTGSGFTGTHCETLMPLCWSKPCHNNATCEDSVDNYTCHCWPGYTGAQCEIDLNECNSNPCQSNGECVELSSEKQYGRITGLPSSFSYHEASGYVCICQPGFTGIHCEEDVNECSSNPCQNGGTCENLPGNYTCHCPFDNLSRTFYGGRDCSDILLGCTHQQCLNNGTCIPHFQDGQHGFSCLCPSGYTGSLCEIATTLSFEGDGFLWVKSGSVTTKGSVCNIALRFQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKIDWNHITLENISSGSSLNVKAGCVRKDWCESQPCQSRGRCINLWLSYQCDCHRPYEGPNCLREYVAGRFGQDDSTGYVIFTLDESYGDTISLSMFVRTLQPSGLLLALENSTYQYIRVWLERGRLAMLTPNSPKLVVKFVLNDGNVHLISLKIKPYKIELYQSSQNLGFISASTWKIEKGDVIYIGGLPDKQETELNGGFFKGCIQDVRLNNQNLEFFPNPTNNASLNPVLVNVTQGCAGDNSCKSNPCHNGGVCHSRWDDFSCSCPALTSGKACEEVQWCGFSPCPHGAQCQPVLQGFECIANAVFNGQSGQILFRSNGNITRELTNITFGFRTRDANVIILHAEKEPEFLNISIQDSRLFFQLQSGNSFYMLSLTSLQSVNDGTWHEVTLSMTDPLSQTSRWQMEVDNETPFVTSTIATGSLNFLKDNTDIYVGDRAIDNIKGLQGCLSTIEIGGIYLSYFENVHGFINKPQEEQFLKISTNSVVTGCLQLNVCNSNPCLHGGNCEDIYSSYHCSCPLGWSGKHCELNIDECFSNPCIHGNCSDRVAAYHCTCEPGYTGVNCEVDIDNCQSHQCANGATCISHTNGYSCLCFGNFTGKFCRQSRLPSTVCGNEKTNLTCYNGGNCTEFQTELKCMCRPGFTGEWCEKDIDECASDPCVNGGLCQDLLNKFQCLCDVAFAGERCEVDLADDLISDIFTTIGSVTVALLLILLLAIVASVVTSNKRATQGTYSPSRQEKEGSRVEMWNLMPPPAMERLI

SEQ ID NO: 9 - protein, 1003 aa, Homo sapiens
MFGARTHGFHILMAMLIGIHCEEDVNECSSNPCQNGGTCENLPGNYTCHCPFDNLSRTFYGGRDCSDILLGCTHQQCLNNGTCIPHFQDGQHGFSCLCPSGYTGSLCEIATTLSFEGDGFLWVKSGSVTTKGSVCNIALRFQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKIDWNHITLENISSGSSLNVKAGCVRKDWCESQPCQSRGRCINLWLSYQCDCHRPYEGPNCLREYVAGRFGQDDSTGYVIFTLDESYGDTISLSMFVRTLQPSGLLLALENSTYQYIRVWLERGRLAMLTPNSPKLVVKFVLNDGNVHLISLKIKPYKIELYQSSQNLGFISASTWKIEKGDVIYIGGLPDKQETELNGGFFKGCIQDVRLNNQNLEFFPNPTNNASLNPVLVNVTQGCAGDNSCKSNPCHNGGVCHSRWDDFSCSCPALTSGKACEEVQWCGFSPCPHGAQCQPVLQGFECIANAVFNGQSGQILFRSNGNITRELTNITFGFRTRDANVIILHAEKEPEFLNISIQDSRLFFQLQSGNSFYMLSLTSLQSVNDGTWHEVTLSMTDPLSQTSRWQMEVDNETPFVTSTIATGSLNFLKDNTDIYVGDRAIDNIKGLQGCLSTIEIGGIYLSYFENVHGFINKPQEEQFLKISTNSVVTGCLQLNVCNSNPCLHGGNCEDIYSSYHCSCPLGWSGKHCELNIDECFSNPCIHGNCSDRVAAYHCTCEPGYTGVNCEVDIDNCQSHQCANGATCISHTNGYSCLCFGNFTGKFCRQSRLPSTVCGNEKTNLTCYNGGNCTEFQTELKCMCRPGFTGEWCEKDIDECASDPCVNGGLCQDLLNKFQCLCDVAFAGERCEVDVSSLSFYVSLLFWQNLFQLLSYLILRMNDEPVVEWGEQEDY